(19) United States
(12) Patent Application Publication
(10) Pub. No.: US 2023/0317236 A1
(43) Pub. Date: Oct. 5, 2023

(54) REHABILITATION ASSISTANCE APPARATUS, REHABILITATION ASSISTANCE SYSTEM, REHABILITATION ASSISTANCE METHOD, AND RECORDING MEDIUM

(71) Applicant: NEC Corporation, Minato-ku, Tokyo (JP)

(72) Inventors: Yuji OHNO, Tokyo (JP); Makoto Yasukawa, Tokyo (JP); Yuki Kosaka, Tokyo (JP); Masahiro Kubo, Tokyo (JP)

(73) Assignee: NEC Corporation, Minato-ku, Tokyo (JP)

(21) Appl. No.: 18/023,251

(22) PCT Filed: Sep. 30, 2020
(86) PCT No.: PCT/JP2020/037116

§ 371 Date: Feb. 24, 2023

(51) Int. Cl. G16H 20/30; G06T 11/00; G06T 7/00; G16H 50/30; G16H 40/67; A63B 24/00; A63B 71/06

(52) U.S. Cl. CPC G16H 20/30; G06T 11/00; G06T 7/0012; G16H 50/30; G16H 40/67; A63B 24/0062; A63B 24/0075; A63B 71/0619; G06T 2207/30004; G06T 2210/41; A63B 2230/062; A63B 2230/425; A63B 2230/305; A63B 2230/625; A63B 2024/0065; A63B 2024/0068

(57) ABSTRACT

A rehabilitation assistance apparatus 1 includes: an acquisition unit 211 configured to acquire at least one of an image of a subject who is doing exercise for rehabilitation, audio of the subject, and biological information of the subject; a calculation unit 212 configured to calculate a fatigue level of the subject based on at least one of the image, the audio, and the biological information; a generation unit 213 configured to generate feedback information related to the rehabilitation, based on the fatigue level; and an output unit 23 configured to output the feedback information.

### TECHNICAL FIELD

The present disclosure relates to a technical field of a rehabilitation assistance apparatus, a rehabilitation assistance system, a rehabilitation assistance method, and a recording medium capable of assisting rehabilitation.

### BACKGROUND ART

Patent Literature 1 describes an example of a rehabilitation assistance apparatus for assisting rehabilitation. Patent Literature 1 describes the rehabilitation assistance apparatus providing an interface that, by using a depth sensor, estimates the posture of a subject exercising for rehabilitation and presents a muscle with which the subject is able to easily exert tension and which is selected from among a group of muscles that can drive a joint that is not functioning well.

Other background art references relevant to the present disclosure are listed below as Patent Literatures 2 to 6.

### CITATION LIST

### Patent Literature

Patent Literature 1
Japanese Patent Laid-Open No. 2016-035651
Patent Literature 2
U.S. Patent Application No. 2019/0295436
Patent Literature 3
Japanese Patent Laid-Open No. 2015-219881
Patent Literature 4
Japanese Patent Laid-Open No. 2020-057082
Patent Literature 5
Japanese Patent Laid-Open No. 2018-033621
Patent Literature 6
Japanese Patent Laid-Open No. 2016-185317

### SUMMARY

### Technical Problem

When a subject does exercise for rehabilitation, it is desirable to have the subject do exercise having an appropriate load. However, Patent Literature 1 and the like do not describe the function of a rehabilitation assistance apparatus to determine whether or not the load of an exercise performed by the subject is an appropriate load. For this reason, the rehabilitation assistance apparatus described in Patent Literature 1 and the like have a technical problem where it is difficult to assist the rehabilitation in such a manner that the load of the exercise performed by the subject (i.e., the load imposed on the subject) and safety of the subject are satisfactorily balanced.

It is an object of the present disclosure to provide a rehabilitation assistance apparatus, a rehabilitation assistance system, a rehabilitation assistance method, and a recording medium that are capable of solving the abovementioned technical problem. As an example, it is an object of the present disclosure to provide a rehabilitation assistance apparatus, a rehabilitation assistance system, a rehabilitation assistance method, and a recording medium that are capable of assisting rehabilitation in such a manner that the load of the exercise performed by the subject for rehabilitation and safety of the subject are satisfactorily balanced.

### Solution to Problem

A rehabilitation assistance apparatus according to one aspect of the present disclosure includes: an acquisition unit configured to acquire at least one of an image of a subject who is doing exercise for rehabilitation, audio of the subject, and biological information of the subject; a calculation unit configured to calculate a fatigue level of the subject based on at least one of the image, the audio, and the biological information; a generation unit configured to generate feedback information related to the rehabilitation, based on the fatigue level; and an output unit configured to output the feedback information.

A rehabilitation assistance system according to one aspect of the present disclosure includes a subject terminal usable by a subject who is doing exercise for rehabilitation; an assistant terminal usable by an assistant who assists the rehabilitation; and a rehabilitation assistance apparatus, wherein the rehabilitation assistance apparatus includes: an acquisition unit configured to acquire at least one of an image of the subject, audio of the subject, and biological information of the subject; a calculation unit configured to calculate a fatigue level of the subject based on at least one of the image, the audio, and the biological information; a generation unit configured to generate feedback information related to the rehabilitation, based on the fatigue level; and an output unit configured to output the feedback information to at least one of the subject terminal and the assistant terminal.

A rehabilitation assistance method according to one aspect of the present disclosure includes acquiring at least one of an image of a subject who is doing exercise for rehabilitation, audio of the subject, and biological information of the subject; calculating a fatigue level of the subject based on at least one of the image, the audio, and the biological information; generating feedback information related to the rehabilitation, based on the fatigue level; and outputting the feedback information.

A recording medium according to one aspect of the present disclosure is a recording medium having recorded thereon a computer program configured to cause a computer to execute: acquiring at least one of an image of a subject who is doing exercise for rehabilitation, audio of the subject, and biological information of the subject; calculating a fatigue level of the subject based on at least one of the image, the audio, and the biological information; generating feedback information related to the rehabilitation, based on the fatigue level; and outputting the feedback information.

### Effect

The rehabilitation assistance apparatus, the rehabilitation assistance system, the rehabilitation assistance method, and the recording medium described above are able to assist the rehabilitation in such a manner that the load of the exercise performed by the subject for rehabilitation and safety of the subject are satisfactorily balanced.

### BRIEF DESCRIPTION OF DRAWINGS

FIG. 1 is a block diagram illustrating an overall configuration of a rehabilitation assistance system of an example embodiment of the present disclosure.

FIG. 2 is a block diagram illustrating a configuration of a patient terminal in the present example embodiment.

FIG. 3 is a block diagram illustrating a configuration of a rehabilitation assistance server in the present example embodiment.

FIG. 4 is a block diagram illustrating a configuration of an assistant terminal in the present example embodiment.

FIG. 5 is a plan view illustrating an output example (a display example) of an image of a patient realized by an output apparatus of the assistant terminal.

FIG. 6 is a flowchart illustrating a flow in a rehabilitation assistance operation performed by the rehabilitation assistance system.

FIG. 7 is a plan view illustrating an output example (a display example) of patient feedback (FB) information including exercise menu information realized by another output apparatus.

FIG. 8 is a plan view illustrating an output example (a display example) of the patient FB information including rest information realized by the output apparatus.

FIG. 9 is a plan view illustrating an output example (a display example) of the patient FB information including cheering information realized by the output apparatus.

FIG. 10 is a plan view illustrating an output example (a display example) of assistant FB information including specific patient information realized by the output apparatus.

FIG. 11 is a plan view illustrating a user interface used by an assistant to input advice.

FIG. 12 is another plan view illustrating the user interface used by the assistant to input advice.

FIG. 13 is a plan view illustrating another user interface used by the assistant to designate an exercise menu.

FIG. 14 is a block diagram illustrating a configuration of a rehabilitation assistance server in a first modification.

FIG. 15 is a plan view illustrating an output example (a display example) of the patient FB information including score information realized by the output apparatus.

### DESCRIPTION OF EXAMPLE EMBODIMENTS

The following will describe example embodiments of a rehabilitation assistance apparatus, a rehabilitation assistance system, a rehabilitation assistance method, and a recording medium, with reference to the drawings. The following will explain a rehabilitation assistance system SYS to which the example embodiments of the rehabilitation assistance apparatus, the rehabilitation assistance system, the rehabilitation assistance method, and the recording medium are applied. The rehabilitation assistance system SYS may be adopted, for example, at an institution (hereinafter, “rehabilitation institution”) that provides a service related to rehabilitation for a subject who does exercise for rehabilitation. Examples of the rehabilitation institution include medical institutions. In the present example embodiment, examples of the medical institutions may include, not only institutions that perform medical practice (e.g., at least one of a hospital and a clinic), but also arbitrary institutions (e.g., at least one of a geriatric nursing healthcare facility, an elderly welfare facility, and a home-visit rehabilitation business) that provide a medical service related to rehabilitation without performing medical practice.

### (1) A CONFIGURATION OF THE REHABILITATION ASSISTANCE SYSTEM SYS

To begin with, a configuration of the rehabilitation assistance system SYS in the present example embodiment will be explained.

(1-1) An Overall Configuration of the Rehabilitation Assistance System SYS

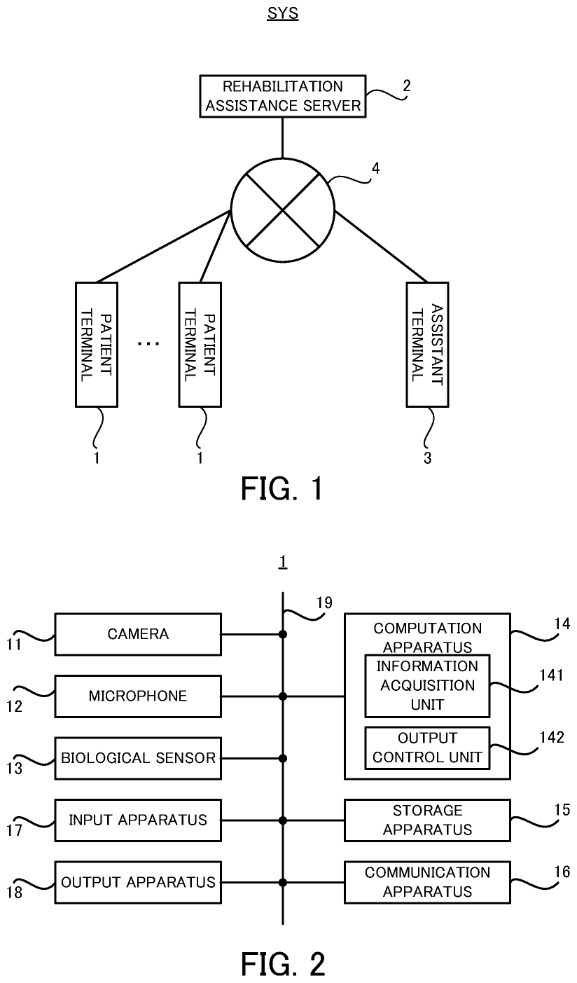

First, an overall configuration of the rehabilitation assistance system SYS in the present example embodiment will be explained, with reference to FIG. 1. FIG. 1 is a block diagram illustrating the overall configuration of the rehabilitation assistance system SYS in the present example embodiment.

The rehabilitation assistance system SYS performs a rehabilitation assistance operation for assisting rehabilitation performed by a subject. For example, the rehabilitation assistance system SYS may assist the subject who is doing exercise for rehabilitation, by performing the rehabilitation assistance operation. For example, the rehabilitation assistance system SYS may assist an assistant who assists the subject doing exercise for rehabilitation (i.e., who assists the rehabilitation), by performing the rehabilitation assistance operation.

In the present example embodiment, the rehabilitation assistance system SYS assists the rehabilitation while the subject is doing exercise in a place that is out of sight of the assistant (i.e., in a place to which the assistant is not able to pay sufficient attention). Examples of the place that is out of sight of the assistant may include a place (e.g., the residence of the patient) different from the facility of the rehabilitation institution. Examples of the place that is out of sight of the assistant may include a place (e.g., a private room of the subject) where the assistant is not present within the facility of the rehabilitation institution. However, the rehabilitation assistance system SYS may also assist the rehabilitation while the subject is doing exercise in a place that is within sight of the assistant.

As explained above, examples of the rehabilitation institution adopting the rehabilitation assistance system SYS include medical institutions. In that situation, examples of the subject doing the exercise for rehabilitation include a patient at a medical institution. In the present example embodiment, the patient may denote a person receiving a medical service represented by rehabilitation at a medical institution. In the following description, an example will be explained in which the subject doing the exercise for rehabilitation is a patient. However, the subject doing the exercise for rehabilitation is not limited to a patient at a medical institution.

The assistant is a medical provider engaged in the rehabilitation of the patient (the subject). In the present example embodiment, the medical provider is, typically, a physical therapist. However, examples of the medical provider may include at least one of an occupational therapist and a speech pathologist. Examples of the medical provider may include people in general engaged in medicine, which represents activities aiming at least one of the following: treating diseases; preventing diseases; maintaining health; recovering health; and promoting health. More specifically, examples of the medical provider may include at least one of the following: a medical doctor; paramedic staff, and other people engaged in medicine. A single assistant may assist a single patient. Alternatively, a single assistant may assist multiple patients.

To assist the rehabilitation, the rehabilitation assistance system SYS monitors the state of the patient in at least a part of the time period during which the patient is doing the exercise. Further, based on the monitored state of the patient, the rehabilitation assistance system SYS presents (i.e., outputs) feedback information (hereinafter, “FB information”) related to the rehabilitation to the patient. Alternatively, the rehabilitation assistance system SYS may present (i.e., output) the FB information to the assistant in addition to or in place of the patient. In this situation, the FB information may denote arbitrary information that is desirable to be presented (desirable to be communicated) to at least one of the patient and the assistant. For example, the FB information may include arbitrary information that is desirable to be presented to at least one of the patient and the assistant, for the purpose of enhancing advantageous effects of the rehabilitation. For example, the FB information may include arbitrary information that is desirable to be presented to at least one of the patient and the assistant, for the purpose of maintaining safety of the patient doing the exercise. For example, the FB information may include arbitrary information that is needed by at least one of the patient and the assistant, for the purpose of enhancing advantageous effects of the rehabilitation. For example, the FB information may include arbitrary information that is needed by at least one of the patient and the assistant, for the purpose of maintaining safety of the patient doing the exercise. As explained in detail later, in order to enhance the advantageous effects of the rehabilitation and/or to maintain safety of the patient doing the exercise, it is preferable that the load of the exercise performed by the patient (i.e., the load imposed on the patient by the exercise) is an appropriate load with which the advantageous effects of the rehabilitation can properly be enjoyed. For this reason, for example, the FB information may include arbitrary information that assists the patient so as to do exercise having an appropriate load. For example, the FB information may include arbitrary information that assists the assistant, so that the assistant is able to cause the patient to do exercise having an appropriate load. In the following sections, the FB information presented to the patient (i.e., FB information for the patient) and the FB information presented to the assistant (i.e., FB information for the assistant) will be distinguished from each other by being referred to as “patient FB information” and “assistant FB information”, respectively.

To perform the rehabilitation assistance operation, as shown in FIG. 1, the rehabilitation assistance system SYS includes at least one patient terminal 1, a rehabilitation assistance server 2 serving as a specific example of the “rehabilitation assistance apparatus”, and at least one assistant terminal 3. In the example shown in FIG. 1, the rehabilitation assistance system SYS includes a plurality of patient terminals 1 and a single assistant terminal 3. However, the rehabilitation assistance system SYS may include a single patient terminal 1. The rehabilitation assistance system SYS may include a plurality of assistant terminals 3. The patient terminals 1, the rehabilitation assistance server 2, and the assistant terminal 3 are able to communicate with one another via a communication network 4. The communication network 4 may include a wired communication network and may include a wireless communication network.

Each of the patient terminals 1 is an information processing apparatus usable by a patient. When the rehabilitation assistance system SYS includes a plurality of patient terminals 1, the plurality of patient terminals 1 may be a plurality of information processing apparatuses that are usable by a plurality of mutually-different patients, respectively. As any of the patient terminals 1, it is possible to use an information processing apparatus (e.g., at least one of a personal computer, a tablet terminal, and a smartphone) loaned to the patient by the rehabilitation institution. As any of the patient terminals 1, it is possible to use an information processing apparatus (e.g., at least one of a personal computer, a tablet terminal, and a smartphone) owned by the patient. As any of the patient terminals 1, it is possible to use a combination of a plurality of information processing apparatuses (e.g., a combination of a smartphone and a smartwatch including a biological sensor 13 (explained later)). Each of the patient terminals 1 is capable of monitoring the state of the patient. Further, each of the patient terminals 1 is capable of transmitting patient state information indicating the monitored state of the patient to the rehabilitation assistance server 2 via the communication network 4. Further, each of the patient terminals 1 is capable of receiving the patient FB information transmitted by the rehabilitation assistance server 2 to the patient terminal 1 via the communication network 4. Furthermore, each of the patient terminals 1 is capable of presenting the received patient FB information to the patient. As a result, the patient is able to recognize the patient FB information.

The rehabilitation assistance server 2 is capable of receiving the patient state information transmitted by any of the patient terminals 1 to the rehabilitation assistance server 2 via the communication network 4. Based on the patient state information, the rehabilitation assistance server 2 is capable of generating at least one of the patient FB information and the assistant FB information. The rehabilitation assistance server 2 is capable of transmitting the generated patient FB information to relevant one or more of the patient terminals 1 via the communication network 4. The rehabilitation assistance server 2 is capable of transmitting the generated assistant FB information to the assistant terminal 3 via the communication network 4.

The rehabilitation assistance server 2 may be, for example, an information processing apparatus (e.g., a personal computer) owned by a medical institution including the rehabilitation institution. The rehabilitation assistance server 2 may be, for example, an information processing apparatus installed in a facility of the medical institution. The rehabilitation assistance server 2 may be, for example, an information processing apparatus installed on the outside of facilities of the medical institution. The rehabilitation assistance server 2 may be, for example, a virtual information processing apparatus installed in a cloud environment.

The assistant terminal 3 is an information processing apparatus usable by the assistant. When the rehabilitation assistance system SYS includes a plurality of assistant terminals 3, the plurality of assistant terminals 3 may be a plurality of information processing apparatuses that are usable by a plurality of mutually-different assistants, respectively. As the assistant terminal 3, it is possible to use an information processing apparatus (e.g., at least one of a personal computer, a tablet terminal, and a smartphone) installed in the rehabilitation institution. As the assistant terminal 3, it is possible to use an information processing apparatus (e.g., at least one of a personal computer, a tablet terminal, and a smartphone) loaned to the assistant by the rehabilitation institution. As the assistant terminal 3, it is possible to use an information processing apparatus (e.g., at least one of a personal computer, a tablet terminal, and a smartphone) owned by the assistant. The assistant terminal 3 is capable of receiving the assistant FB information transmitted by the rehabilitation assistance server 2 to the assistant terminal 3 via the communication network 4. Furthermore, the assistant terminal 3 is capable of presenting the received assistant FB information to the assistant. As a result, the assistant is able to recognize the assistant FB information.

(1-2) A Configuration of the Patient Terminals 1

Next, a configuration of the patient terminals 1 will be explained, with reference to FIG. 2. FIG. 2 is a block diagram illustrating the configuration of any of the patient terminals 1.

As shown in FIG. 2, the patient terminal 1 includes a camera 11, a microphone 12, the biological sensor 13, a computation apparatus 14, a storage apparatus 15, a communication apparatus 16, an input apparatus 17, and an output apparatus 18. However, the patient terminal 1 may include all of the camera 11, the microphone 12, and the biological sensor 13 or may include at least one selected from among the camera 11, the microphone 12, and the biological sensor 13. The patient terminal 1 does not necessarily have to include the input apparatus 17. The camera 11, the microphone 12, the biological sensor 13, the computation apparatus 14, the storage apparatus 15, the communication apparatus 16, the input apparatus 17, and the output apparatus 18 may be connected together via a data bus 19.

As explained above, it is possible to use a combination of a plurality of information processing apparatuses as the patient terminal 1. In that situation, the patient terminal 1 may be realized with a combination of: a first information processing apparatus including one or more selected from among the camera 11, the microphone 12, the biological sensor 13, the computation apparatus 14, the storage apparatus 15, the communication apparatus 16, the input apparatus 17, and the output apparatus 18; and a second information processing apparatus including one or more other members selected from among the camera 11, the microphone 12, the biological sensor 13, the computation apparatus 14, the storage apparatus 15, the communication apparatus 16, the input apparatus 17, and the output apparatus 18. For example, the patient terminal 1 may be realized with a combination of: an information processing apparatus (e.g., a smartphone, a tablet terminal, or a personal computer) including the camera 11 and the microphone 12; another information processing apparatus (e.g., a wearable apparatus such as a smartwatch) including the biological sensor 13; and yet another information processing apparatus (e.g., a smartphone, a tablet terminal, or a personal computer) including the computation apparatus 14, the storage apparatus 15, the communication apparatus 16, the input apparatus 17, and the output apparatus 18.

The camera 11 is an imaging apparatus capable of imaging the patient. The camera 11 generates an image taken of the patient, by imaging the patient. Patient image information represented by the generated image is transmitted, as at least a part of the patient state information, from the patient terminal 1 to the rehabilitation assistance server 2. In the present example embodiment, the image may include a still image. Alternatively, the image may include a video (i.e., time series data including a plurality of still images generated by imaging the patient at a plurality of mutually-different consecutive points in time) in addition to or in place of the still image.

The microphone 12 is a recording apparatus capable of recording audio of the patient. Patient audio information represented by the audio of the patient recorded by the microphone 12 is transmitted, as at least a part of the patient state information, from the patient terminal 1 to the rehabilitation assistance server 2.

The biological sensor 13 is a detection apparatus capable of detecting biological information of the patient (hereinafter, “patient biological information”). The biological sensor 13 may include, for example, a heartbeat sensor capable of detecting information related to heartbeats serving as an example of the patient biological information. The biological sensor 13 may include, for example, a respiration sensor capable of detecting information related to respiration serving as an example of the patient biological information. The biological sensor 13 may include, for example, a blood pressure sensor capable of detecting information related to blood pressure serving as an example of the patient biological information. The biological sensor 13 may include, for example, perspiration sensor capable of detecting information related to perspiration of the patient serving as an example of the patient biological information. The biological sensor 13 may include, for example, a motion sensor (typically, at least one of an acceleration sensor and an angular velocity sensor) capable of detecting information related to movements of patient serving as an example of the patient biological information. The patient biological information detected by the biological sensor 13 is transmitted, as at least a part of the patient state information, from the patient terminal 1 to the rehabilitation assistance server 2.

In this situation, it is possible to generate the patient biological information by analyzing the image represented by the patient image information. For example, it is possible to generate the information related to heartbeats, by analyzing the image represented by the patient image information. In that situation, the patient image information may be used as the patient biological information. When the patient image information is used as the patient biological information, the patient terminal 1 does not necessarily have to include the biological sensor 13.

The computation apparatus 14 includes a Central Processing Unit (CPU), for example. The computation apparatus 14 reads a computer program. For example, the computation apparatus 14 may read a computer program stored in the storage apparatus 15. For example, the computation apparatus 14 may read a computer program stored in a computer-readable non-transitory recording medium, by using a recording medium reading apparatus (not shown). The computation apparatus 14 may acquire (i.e., may download or may read) a computer program from an apparatus (not shown) disposed on the outside of the patient terminal 1, via the communication apparatus 16. The computation apparatus 14 executes the read computer program. As a result, realized in the computation apparatus 14 are logical functional blocks for executing operations (e.g., at least a part of the rehabilitation assistance operation) that are to be performed by the patient terminal 1. In other words, the computation apparatus 14 is capable of functioning as a controller for realizing the logical functional blocks for executing the operations that are to be performed by the patient terminal 1.

FIG. 2 shows an example of the logical functional blocks realized in the computation apparatus 14 for performing at least a part of the rehabilitation assistance operation. As shown in FIG. 2, realized in the computation apparatus 14 are an information acquisition unit 141 and an output control unit 142. The information acquisition unit 141 acquires at least one of the patient image information, the patient audio information, and the patient biological information from at least one of the camera 11, the microphone 12, and the biological sensor 13, respectively. By using the communication apparatus 16, the information acquisition unit 141 transmits the patient state information including at least one of the patient image information, the patient audio information, and the patient biological information that was acquired, to the rehabilitation assistance server 2. By using the communication apparatus 16, the output control unit 142 acquires (i.e., receives) the patient FB information from the rehabilitation assistance server 2. The output control unit 142 controls the output apparatus 18 so as to present (i.e., output) the acquired patient FB information to the patient.

In this situation, at least one of the information acquisition unit 141 and the output control unit 142 may be realized by at least one of an application and a browser installed as software in the patient terminal 1. In that situation, the information acquisition unit 141 may acquire at least one of the patient image information, the patient audio information, and the patient biological information, via at least one of the application and the browser. The output control unit 142 may control the output apparatus 18 so as to present the patient FB information to the patient, via at least one of the application and the browser.

The storage apparatus 15 is capable of storing therein desired data. For example, the storage apparatus 15 may temporarily store therein the computer program to be executed by the computation apparatus 14. The storage apparatus 15 may temporarily store therein data temporarily used by the computation apparatus 14 while the computation apparatus 14 is executing the computer program. The storage apparatus 15 may store therein data to be saved by the patient terminal 1 for a long period of time. Further, the storage apparatus 15 may include at least one of the following: a Random Access Memory (RAM), a Read Only Memory (ROM), a hard disk apparatus, a magneto optical disk apparatus, a Solid State Drive (SSD), and a disk array apparatus. In other words, the storage apparatus 15 may include a non-transitory recording medium.

The communication apparatus 16 is capable of transmitting information to the rehabilitation assistance server 2 via the communication network 4. In the present example embodiment, as explained above, the communication apparatus 16 is capable, under the control of the information acquisition unit 141, of transmitting the patient state information including at least one of the patient image information, the patient audio information, and the patient biological information, to the rehabilitation assistance server 2. Further, the communication apparatus 16 is capable of receiving information transmitted by the rehabilitation assistance server 2 via the communication network 4. In the present example embodiment, as explained above, the communication apparatus 16 is capable of receiving the patient FB information transmitted by the rehabilitation assistance server 2 under the control of the output control unit 142.

The input apparatus 17 is an apparatus that receives an input of information to the patient terminal 1 from the outside of the patient terminal 1. For example, the input apparatus 17 may include an operation apparatus (e.g., at least one of a keyboard, a mouse, and a touch panel) operable by the patient. For example, the input apparatus 17 may include an audio input apparatus (e.g., a microphone) that receives audio of the patient as an input. In that situation, the audio of the patient may be used as an audio input designating specifics of an operation performed by the patient. In an example, the microphone 12 may be used as the input apparatus 17. For example, the input apparatus 17 may include a reading apparatus capable of reading information recorded as data in a recording medium that can externally be attached to the patient terminal 1.

The output apparatus 18 is an apparatus that outputs information to the outside of the patient terminal 1. For example, the output apparatus 18 may output the information as an image. In other words, the output apparatus 18 may include a display apparatus (a so-called display) capable of displaying the image representing the information to be output. In that situation, the output apparatus 18 may present the patient FB information to the patient, by displaying (i.e., outputting) the patient FB information as an image. As a result, the patient is able to recognize the patient FB information visually. As another example, the output apparatus 18 may output the information as audio. In other words, the output apparatus 18 may include an audio output apparatus (a so-called speaker) capable of outputting the audio. In that situation, the output apparatus 18 may present the patient FB information to the patient, by outputting the patient FB information as audio. As a result, the patient is able to recognize the patient FB information auditorily. As yet another example, the output apparatus 18 may output the information on a sheet of paper. In other words, the output apparatus 18 may include a printing apparatus (a so-called printer) capable of printing desired information on a sheet of paper. In that situation, the output apparatus 18 may present the patient FB information to the patient, by printing (i.e., outputting) the patient FB information on a sheet of paper. As a result, the patient is able to recognize the patient FB information visually. In the following description, to simplify the explanation, an example will be explained in which the output apparatus 18 is a display apparatus.

(1-3) A Configuration of the Rehabilitation Assistance Server 2

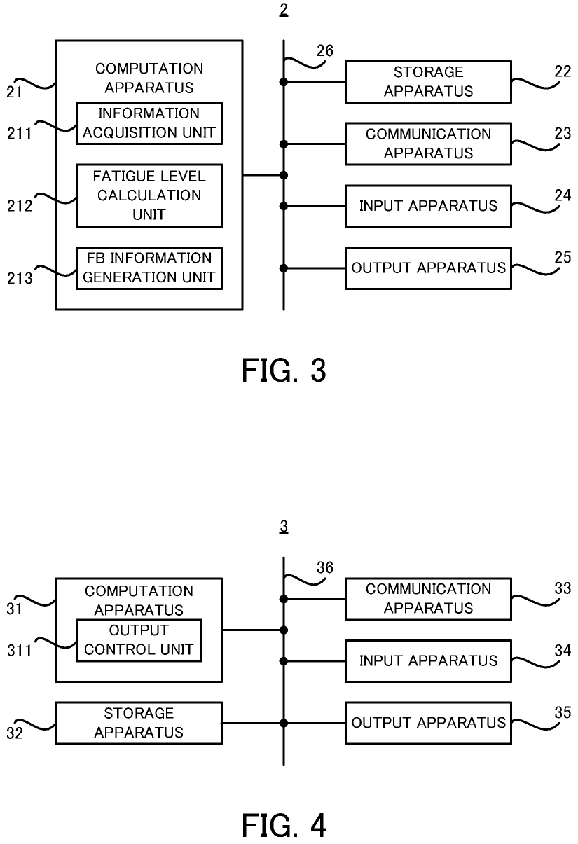

Next, a configuration of the rehabilitation assistance server 2 will be explained, with reference to FIG. 3. FIG. 3 is a block diagram illustrating the configuration of the rehabilitation assistance server 2.

As shown in FIG. 3, the rehabilitation assistance server 2 includes a computation apparatus 21, a storage apparatus 22, and a communication apparatus 23. Further, the rehabilitation assistance server 2 may include an input apparatus 24 and an output apparatus 25. However, the rehabilitation assistance server 2 may omit at least one of the input apparatus 24 and the output apparatus 25. The computation apparatus 21, the storage apparatus 22, the communication apparatus 23, the input apparatus 24, and the output apparatus 25 may be connected together via a data bus 26.

The computation apparatus 21 includes a CPU, for example. The computation apparatus 21 reads a computer program. For example, the computation apparatus 21 may read a computer program stored in the storage apparatus 22. For example, the computation apparatus 21 may read a computer program stored in a computer-readable non-transitory recording medium, by using a recording medium reading apparatus (not shown). The computation apparatus 21 may acquire (i.e., may download or may read) a computer program from an apparatus (not shown) disposed on the outside of the rehabilitation assistance server 2, via the communication apparatus 23. The computation apparatus 21 executes the read computer program. As a result, realized in the computation apparatus 21 are logical functional blocks for executing operations (e.g., at least a part of the rehabilitation assistance operation) that are to be performed by the rehabilitation assistance server 2. In other words, the computation apparatus 21 is capable of functioning as a controller for realizing the logical functional blocks for executing the operations that are to be performed by the rehabilitation assistance server 2.

FIG. 3 shows an example of the logical functional blocks realized in the computation apparatus 21 for executing at least a part of the rehabilitation assistance operation. As shown in FIG. 3, realized in the computation apparatus 21 are an information acquisition unit 211 which is a specific example of the “acquisition unit”, a fatigue level calculation unit 212 which is a specific example of the “calculation unit”, and an FB information generation unit 213 which is a specific example of the “generation unit”. By using the communication apparatus 23, the information acquisition unit 211 acquires (i.e., receives) the patient state information from any of the patient terminals 1. The fatigue level calculation unit 212 calculates a fatigue level of the patient based on the patient state information. The fatigue level will be explained in detail later. The FB information generation unit 213 generates the FB information (i.e., at least one of the patient FB information and the assistant FB information) based on the fatigue level calculated by the fatigue level calculation unit 212. By using the communication apparatus 23, the FB information generation unit 213 transmits (i.e., outputs) the generated patient FB information to a relevant one of the patient terminals 1. By using the communication apparatus 23, the FB information generation unit 213 transmits (i.e., outputs) the generated assistant FB information to the assistant terminal 3.

The storage apparatus 22 is capable of storing therein desired data. For example, the storage apparatus 22 may temporarily store therein the computer program to be executed by the computation apparatus 21. The storage apparatus 22 may temporarily store therein data temporarily used by the computation apparatus 21 while the computation apparatus 21 is executing the computer program. The storage apparatus 22 may store therein data to be saved by the rehabilitation assistance server 2 for a long period of time. Further, the storage apparatus 22 may include at least one of the following: a RAM, a ROM, a hard disk apparatus, a magneto optical disk apparatus, an SSD, and a disk array apparatus. In other words, the storage apparatus 22 may include a non-transitory recording medium.

The communication apparatus 23 is capable of transmitting information to at least one of the patient terminals 1 and the assistant terminal 3, via the communication network 4. In the present example embodiment, as explained above, under the control of the FB information generation unit 213, the communication apparatus 23 is capable of transmitting (i.e., capable of outputting) at least one of the patient FB information and the assistant FB information, to at least one of the patient terminals 1 and the assistant terminal 3, respectively. In this situation, the communication apparatus 23 may be referred to as an output apparatus. Further, the communication apparatus 23 is capable of receiving information transmitted by at least one of the patient terminals 1 and the assistant terminal 3 via the communication network 4. In the present example embodiment, as explained above, under the control of the information acquisition unit 211, the communication apparatus 23 is capable of receiving the patient state information transmitted by any of the patient terminals 1.

The input apparatus 24 is an apparatus that receives an input of information to the rehabilitation assistance server 2 from the outside of the rehabilitation assistance server 2. For example, the input apparatus 24 may include an operation apparatus (e.g., at least one of a keyboard, a mouse, and a touch panel) operable by a user of the rehabilitation assistance server 2. For example, the input apparatus 24 may include a reading apparatus capable of reading information recorded as data in a recording medium that can externally be attached to the rehabilitation assistance server 2.

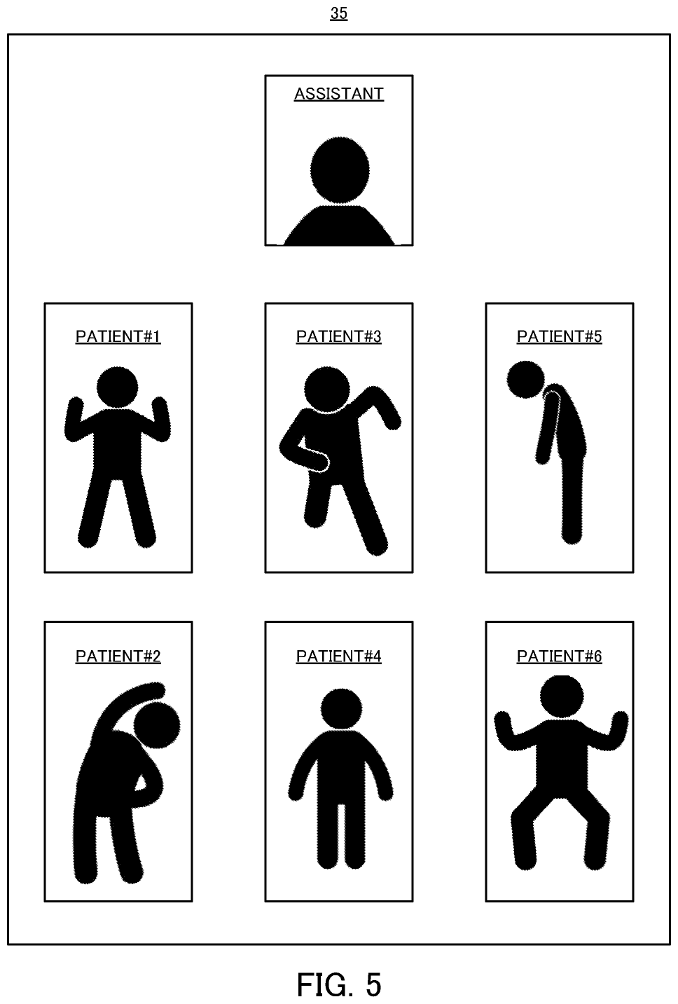

Further, at least one of the patient terminals 1 and the assistant terminal 3 may be used as the input apparatus 24 of the rehabilitation assistance server 2. As an example, the input apparatus 17 of any of the patient terminals 1 may be used as the input apparatus 24 of the rehabilitation assistance server 2. As another example, an input apparatus 34 (see FIG. 4 explained later) of the assistant terminal 3 may be used as the input apparatus 24 of the rehabilitation assistance server 2.

The output apparatus 25 is an apparatus that outputs information to the outside of the rehabilitation assistance server 2. For example, the output apparatus 25 may output the information as an image. In other words, the output apparatus 25 may include a display apparatus (a so-called display) capable of displaying the image representing the information to be output. As another example, the output apparatus 25 may output the information as audio. In other words, the output apparatus 25 may include an audio output apparatus (a so-called speaker) capable of outputting the audio. As yet another example, the output apparatus 25 may output the information on a sheet of paper. In other words, the output apparatus 25 may include a printing apparatus (a so-called printer) capable of printing desired information on a sheet of paper.

Further, at least one of the patient terminals 1 and the assistant terminal 3 may be used as an output apparatus 35 of the rehabilitation assistance server 2. For example, the output apparatus 18 of any of the patient terminals 1 may be used as the output apparatus 25 of the rehabilitation assistance server 2. As another example, the output apparatus 35 (see FIG. 4 explained later) of the assistant terminal 3 may be used as the output apparatus 35 of the rehabilitation assistance server 2.

(1-4) A Configuration of the Assistant Terminal 3

Next, a configuration of the assistant terminal 3 will be explained, with reference to FIG. 4. FIG. 4 is a block diagram illustrating the configuration of the assistant terminal 3.

As shown in FIG. 4, the assistant terminal 3 includes a computation apparatus 31, a storage apparatus 32, and a communication apparatus 33. Further, the assistant terminal 3 may include the input apparatus 34 and the output apparatus 35 serving as a specific example of the “output unit”. However, the assistant terminal 3 may omit at least one of the input apparatus 34 and the output apparatus 35. The computation apparatus 31, the storage apparatus 32, the communication apparatus 33, the input apparatus 34, and the output apparatus 35 may be connected together via a data bus 36.

The computation apparatus 31 includes a CPU, for example. The computation apparatus 31 reads a computer program. For example, the computation apparatus 31 may read a computer program stored in the storage apparatus 32. For example, the computation apparatus 31 may read a computer program stored in a computer-readable non-transitory recording medium, by using a recording medium reading apparatus (not shown). The computation apparatus 31 may acquire (i.e., may download or may read) a computer program from an apparatus (not shown) disposed on the outside of the assistant terminal 3, via the communication apparatus 33. The computation apparatus 31 executes the read computer program. As a result, realized in the computation apparatus 31 are logical functional blocks for executing operations (e.g., at least a part of the rehabilitation assistance operation) that are to be performed by the assistant terminal 3. In other words, the computation apparatus 31 is capable of functioning as a controller for realizing the logical functional blocks for executing the operations that are to be performed by the assistant terminal 3.

FIG. 4 shows an example of the logical functional blocks realized in the computation apparatus 31 for executing at least a part of the rehabilitation assistance operation. As shown in FIG. 4, realized in the computation apparatus 31 is an output control unit 311. By using the communication apparatus 33, the output control unit 311 acquires (i.e., receives) the assistant FB information from the rehabilitation assistance server 2. The output control unit 311 controls the output apparatus 35 so as to present (output) the acquired assistant FB information to the assistant.

In this situation, the output control unit 311 may be realized by at least one of an application and a browser installed as software in the assistant terminal 3. In that situation, the output control unit 311 may control the output apparatus 35 so as to present the assistant FB information to the assistant, via at least one of the application and the browser.

The storage apparatus 32 is capable of storing therein desired data. For example, the storage apparatus 32 may temporarily store therein the computer program to be executed by the computation apparatus 31. The storage apparatus 32 may temporarily store therein data temporarily used by the computation apparatus 31 while the computation apparatus 31 is executing the computer program. The storage apparatus 32 may store therein data to be saved by the assistant terminal 3 for a long period of time. Further, the storage apparatus 32 may include at least one of the following: a RAM, a ROM, a hard disk apparatus, a magneto optical disk apparatus, an SSD, and a disk array apparatus. In other words, the storage apparatus 32 may include a non-transitory recording medium.

The communication apparatus 33 is capable transmitting information to the rehabilitation assistance server 2 via the communication network 4. Further, the communication apparatus 33 is capable of receiving information transmitted by the rehabilitation assistance server 2 via the communication network 4. In the present example embodiment, as explained above, the communication apparatus 33 is capable of receiving the assistant FB information transmitted by the rehabilitation assistance server 2 under the control of the output control unit 311.

The input apparatus 34 is an apparatus that receives an input of information to the assistant terminal 3 from the outside of the assistant terminal 3. For example, the input apparatus 34 may include an operation apparatus (e.g., at least one of a keyboard, a mouse, and a touch panel) operable by the assistant. For example, the input apparatus 34 may include an audio input apparatus (e.g., a microphone) that receives audio of the assistant as an input. In that situation, the audio of the assistant may be used as an audio input designating specifics of an operation performed by the assistant. For example, the input apparatus 34 may include a reading apparatus capable of reading information recorded as data in a recording medium that can externally be attached to the assistant terminal 3.

The output apparatus 35 is an apparatus that outputs information to the outside of the assistant terminal 3. For example, the output apparatus 35 may output the information as an image. In other words, the output apparatus 35 may include a display apparatus (a so-called display) capable of displaying the image representing the information to be output. In that situation, the output apparatus 35 may present the assistant FB information to the assistant, by displaying (i.e., outputting) the assistant FB information as an image. As a result, the assistant is able to recognize the assistant FB information visually. As another example, the output apparatus may output the information as audio. In other words, the output apparatus 35 may include an audio output apparatus (a so-called speaker) capable of outputting the audio. In that situation, the output apparatus 35 may present the assistant FB information to the assistant, by outputting the assistant FB information as audio. As a result, the assistant is able to recognize the assistant FB information auditorily. As yet another example, the output apparatus 35 may output the information on a sheet of paper. In other words, the output apparatus 35 may include a printing apparatus (a so-called printer) capable of printing desired information on a sheet of paper. In that situation, the output apparatus 35 may present the assistant FB information to the assistant, by printing (i.e., outputting) the assistant FB information on a sheet of paper. As a result, the assistant is able to recognize the assistant FB information visually. In the following description, to simplify the explanation, an example will be explained in which the output apparatus 35 is a display apparatus.

When the output apparatus 35 includes a display apparatus, the output apparatus 35 may display images of one or more patients assisted by the assistant (i.e., the images generated by the cameras 11 that imaged the patients). A display example of the images of the patients is shown in FIG. 5. FIG. 5 shows the display example of the images of the patients when the assistant is assisting a plurality of (six in the example shown in FIG. 5) patients. In this situation, via the communication network 4, the communication apparatus 33 may acquire the patient image information from at least one of the patient terminals 1 and the rehabilitation assistance server 2. After that, the output control unit 311 may output the output apparatus 35 so as to display the images represented by the patient image information.

When the output apparatus 35 includes a display apparatus, the output apparatus 35 may display, as shown in FIG. 5, an image of the assistant in addition to the images of the patients. For example, the assistant terminal 3 may include a camera capable of imaging the assistant. The output apparatus 35 may display the image generated by the camera that imaged the assistant.

When the output apparatus 35 includes an audio output apparatus (a speaker), the output apparatus 35 may output audio of any of the patients assisted by the assistant (i.e., the audio of the patient recorded by the microphone 12). In that situation, via the communication network 4, the communication apparatus 33 may acquire the patient audio information from at least one of the patient terminals 1 and the rehabilitation assistance server 2. After that, the output control unit 311 may control the output apparatus 35 so as to output the audio represented by the patient audio information.

When the output apparatus 35 includes an audio output apparatus, the output apparatus 35 may output audio of the assistant, in addition to the audio of any of the patients. For example, the assistant terminal 3 may include a microphone capable of recording the audio of the assistant. The output apparatus 35 may output the audio of the assistant recorded by the microphone.

Although detailed explanations will be omitted for the sake of convenience in the explanations, the output apparatus 18 of any of the patient terminals 1 may also display, similarly to the output apparatus 35, an image of the patient (here, an image of the patient using the particular patient terminal 1) and an image of the assistant. The output apparatus 18 of any of the patient terminals 1 may also output, similarly to the output apparatus 35, audio of the patient (here, audio of the patient using the particular patient terminal 1) and audio of the assistant.

### (2) A REHABILITATION ASSISTANCE OPERATION PERFORMED BY THE REHABILITATION ASSISTANCE SYSTEM SYS

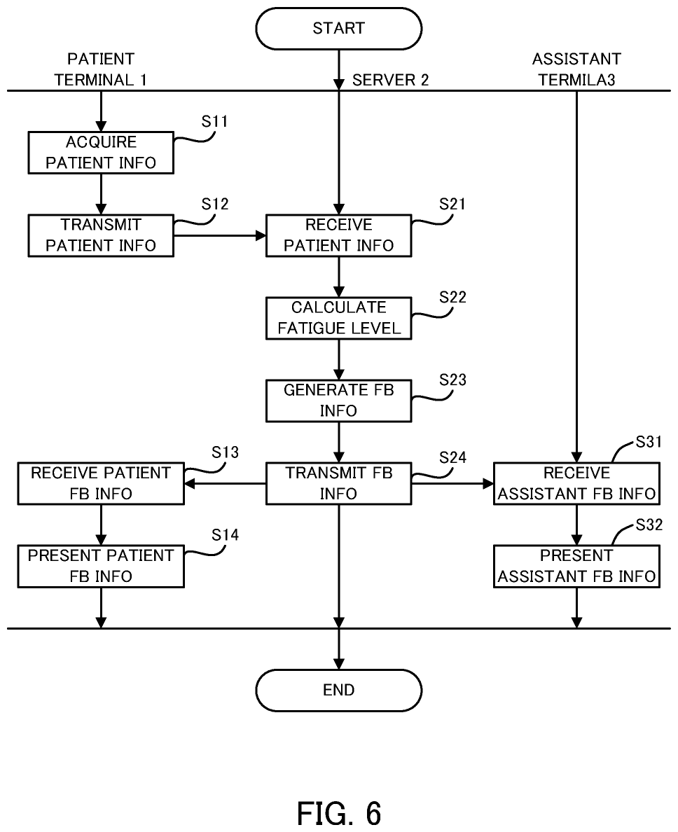

Next, a rehabilitation assistance operation performed by the rehabilitation assistance system SYS will be explained, with reference to FIG. 6. FIG. 6 is a flowchart illustrating a flow in the rehabilitation assistance operation performed by the rehabilitation assistance system SYS.

As shown in FIG. 6, to begin with, a patient terminal 1 acquires patient state information (step S11). More specifically, the information acquisition unit 141 of the patient terminal 1 may acquire the patient image information from the camera 11. The information acquisition unit 141 may acquire the patient audio information from the microphone 12. The information acquisition unit 141 may acquire the patient biological information from the biological sensor 13. In this situation, the patient terminal 1 typically acquires the patient state information in at least a part of the time period during which the patient is doing exercise.

Subsequently, the information acquisition unit 141 of the patient terminal 1 transmits, by using the communication apparatus 16, the patient state information including at least one of the patient image information, the patient audio information, and the patient biological information acquired in step S11, to the rehabilitation assistance server 2 via the communication network 4 (step S12). As a result, the information acquisition unit 211 of the rehabilitation assistance server 2 receives, by using the communication apparatus 23, the patient state information transmitted from the patient terminal 1 via the communication network 4 (step S21). As explained above, when the rehabilitation assistance system SYS includes the plurality of patient terminals 1 respectively corresponding to the plurality of mutually-different patients, the information acquisition unit 211 may receive a plurality of pieces of patient state information respectively transmitted from the plurality of patient terminals 1.

After that, based on the patient state information received in step S21, the rehabilitation assistance server 2 generates the FB information and transmits the generated FB information to at least one of the patient terminal 1 and the assistant terminal 3 (steps S22 through S24). In this situation, in at least a part of the time period during which the patient is doing the exercise, the rehabilitation assistance server 2 may generate the FB information and transmit the generated FB information to at least one of the patient terminal 1 and the assistant terminal 3. Alternatively, in addition to or in place of the part of the time period during which the patient is doing the exercise, the rehabilitation assistance server 2 may generate the FB information and transmit the generated FB information to at least one of the patient terminal 1 and the assistant terminal 3, in at least a part of another time period different from the time period during which the patient is doing the exercise. For example, the rehabilitation assistance server 2 may generate the FB information and transmit the generated FB information to at least one of the patient terminal 1 and the assistant terminal 3, in at least a part of the time period during which the patient is not doing the exercise (e.g., after the patient finishes doing the exercise).

To generate and transmit the FB information, at first, the fatigue level calculation unit 212 of the rehabilitation assistance server 2 calculates a fatigue level of the patient, based on the patient state information (step S22). More specifically, based on the patient state information indicating a state of a single patient, the fatigue level calculation unit 212 calculates a fatigue level of the single patient. When the information acquisition unit 211 has acquired the plurality of piece of patient state information respectively corresponding to the plurality of patients, the fatigue level calculation unit 212 may calculate, based on the plurality of pieces of patient state information, fatigue levels of the plurality of patients respectively corresponding to the plurality of pieces of patient state information.

The fatigue level is an index value indicating a degree of physical fatigue of each of the patients. More specifically, while doing the exercise for rehabilitation, the patient accumulates physical fatigue caused by the exercise. For this reason, the fatigue level may be an index value indicating a degree of physical fatigue of each of the patients caused by the exercise for rehabilitation. In the present example embodiment, an example will be explained in which the fatigue level is an index value that becomes larger as the fatigue of the patient becomes larger. As another example, in some situations, while doing the exercise for rehabilitation, there is a possibility that the patient may accumulate psychological fatigue caused by the exercise. For this reason, the fatigue level may be an index value comprehensively indicating a degree of physical fatigue of the patient and a degree of psychological fatigue of the patient.

The fatigue level calculation unit 212 may calculate a feature value related to the state of each of the patients based on the patient state information and may further calculate the fatigue level based on the calculated feature value. More specifically, based on the feature value related to the state of the patient, the fatigue level calculation unit 212 may determine whether or not a sign (hereinafter, “fatigue sign”) indicating that the patient is fatigued is exhibited in the state of the patient. When it is determined that a fatigue sign is exhibited in the state of the patient, the fatigue level calculation unit 212 may calculate a fatigue level corresponding to the fatigue sign.

In an example, the fatigue level calculation unit 212 may calculate a feature value related to movements of the patient, based on at least one of the patient image information and the patient biological information (in particular, information related to movements of the patient). After that, based on the feature value related to the movements of the patient, the fatigue level calculation unit 212 may determine whether or not a fatigue sign is exhibited in the movements of the patient. Examples of the fatigue sign exhibited in the movements of the patient may include at least one of the following: a sign indicating that the movements of the patient are slower than movements that are naturally expected (i.e., ideal movements); a sign indicating that the movements of the patient are different from the movements that are naturally expected; a sign indicating that the movements of the patient are out of balance; a sign indicating that the patient is wobbling; a sign indicating that the movements of the patient are slower than movements of the patient in the past (at an earlier time on the same day or in an earlier day); and a sign indicating that the movements of the patient are out of balance more than the movements of the patient in the past. In these situations, the fatigue level calculation unit 212 may calculate fatigue levels in such a manner that a fatigue level corresponding to the time when the movements of the patient are slower than the movements that are naturally expected or than the movements of the patient in the past is higher than a fatigue level corresponding to the time when the movements of the patient are not slower than the movements that are naturally expected or than the movements of the patient in the past. The fatigue level calculation unit 212 may calculate a fatigue level in such a manner that the more the movements of the patient are slower than the movements that are naturally expected or than the movements of the patient in the past, the higher is the fatigue level. The fatigue level calculation unit 212 may calculate fatigue levels, in such a manner that a fatigue level corresponding to the time when the movements of the patient are different from the movements that are naturally expected or from the movements of the patient in the past is higher than a fatigue level corresponding to the time when the movements of the patient are not different from the movements that are naturally expected or from the movements of the patient in the past. The fatigue level calculation unit 212 may calculate a fatigue level in such a manner that the more different the movements of the patient are from the movements that are naturally expected or from the movements of the patient in the past, the higher is the fatigue level. The fatigue level calculation unit 212 may calculate fatigue levels, in such a manner that a fatigue level corresponding to the time when the movements of the patient are out of balance is higher than a fatigue level corresponding to the time when the movements of the patient are not out of balance. The fatigue level calculation unit 212 may calculate a fatigue level in such a manner that the more the movements of the patient are out of balance, the higher is the fatigue level. The fatigue level calculation unit 212 may calculate fatigue levels, in such a manner that a fatigue level corresponding to the time when the patient is wobbling is higher than a fatigue level corresponding to the time when the patient is not wobbling. The fatigue level calculation unit 212 may calculate a fatigue level in such a manner that the more wobbly the patient is, the higher is the fatigue level.

In another example, the fatigue level calculation unit 212 may calculate a feature value related to a facial expression of the patient, based on the patient image information. Subsequently, based on the feature value related to the facial expression of the patient, the fatigue level calculation unit 212 may determine whether or not a fatigue sign is exhibited in the facial expression of the patient. Examples of the fatigue sign exhibited in the facial expression may include at least one of the following: a sign indicating that the patient has a vacant expression; a sign indicating that the patient has a painful expression; a sign indicating that the patient is absent-minded; and a sign indicating that the patient's eyes are wandering. In these situations, the fatigue level calculation unit 212 may calculate fatigue levels in such a manner that a fatigue level corresponding to the time when the patient has a vacant expression is higher than a fatigue level corresponding to the time when the patient does not have a vacant expression. The fatigue level calculation unit 212 may calculate fatigue levels in such a manner that a fatigue level corresponding to the time when the patient has a painful expression is higher than a fatigue level corresponding to the time when the patient does not have a painful expression. The fatigue level calculation unit 212 may calculate fatigue levels in such a manner that a fatigue level corresponding to the time when the patient looks absent-minded is higher than a fatigue level corresponding to the time when the patient does not look absent-minded. The fatigue level calculation unit 212 may calculate fatigue levels in such a manner that a fatigue level corresponding to the time when the patient's eyes are wandering is higher than a fatigue level corresponding to the time when the patient's eyes are not wandering.

In yet another example, the fatigue level calculation unit 212 may calculate a feature value related to heartbeats, based on the patient biological information (in particular, information related to heartbeats). The feature value related to the heartbeats may include at least one of a heart rate (i.e., the number of heartbeats per unit time period) and irregularities in the heartbeats. Subsequently, based on the feature value related to the heartbeats, the fatigue level calculation unit 212 may determine whether or not a fatigue sign is exhibited in the heartbeats. Examples of the fatigue sign exhibited in the heartbeats may include at least one of the following: a sign indicating that the heart rate exceeds a tolerated heart rate; a sign indicating that a change amount (e.g., an increase amount) in the heart rate per unit time period exceeds a tolerated heartbeat change amount; an arbitrary sign obtained from a heartbeat fluctuation analysis (a frequency analysis on heartbeat intervals), and a sign corresponding to an arrhythmia. In these situations, the fatigue level calculation unit 212 may calculate fatigue levels in such a manner that a fatigue level corresponding to the time when the heart rate exceeds the tolerated heart rate is higher than a fatigue level corresponding to the time when the heart rate does not exceed the tolerated heart rate. The fatigue level calculation unit 212 may calculate a fatigue level in such a manner that the higher the heart rate is, the higher is the fatigue level. The fatigue level calculation unit 212 may calculate fatigue levels in such a manner that a fatigue level corresponding to the time when a change amount in the heart rate exceeds the tolerated heartbeat change amount is higher than a fatigue level corresponding to the time when a change amount in the heart rate does not exceed the tolerated heartbeat change amount. The fatigue level calculation unit 212 may calculate a fatigue level in such a manner that the more different a change amount in the heart rate is from the tolerated heartbeat change amount, the higher is the fatigue level. The fatigue level calculation unit 212 may calculate a fatigue level based on the heart beat fluctuation analysis. The fatigue level calculation unit 212 may calculate fatigue levels in such a manner that a fatigue level corresponding to the time when an arrythmia is exhibited is higher than a fatigue level corresponding to the time when no arrythmia is exhibited.

In yet another example, the fatigue level calculation unit 212 may calculate a feature value related to respiration, based on at least one of the patient image information, the patient audio information, and the patient biological information (in particular, information related to respiration). Examples of the feature value related to the respiration may include at least one of a respiratory rate (i.e., the number of times of breaths per unit time period) and respiratory irregularities. Subsequently, based on the feature value related to the respiration, the fatigue level calculation unit 212 may determine whether or not a fatigue sign is exhibited in the respiration. Examples of the fatigue sign exhibited in the respiration may include at least one of the following: a sign indicating that the respiratory rate exceeds a tolerated respiratory rate; a sign indicating that a change amount (e.g., an increase amount) in the respiratory rate per unit time period exceeds a tolerated respiratory change amount; and a sign indicating irregular respiration. In these situations, the fatigue level calculation unit 212 may calculate fatigue levels in such a manner that a fatigue level corresponding to the time when the respiratory rate exceeds the tolerated respiratory rate is higher than a fatigue level corresponding to the time when the respiratory rate does not exceed the tolerated respiratory rate. The fatigue level calculation unit 212 may calculate a fatigue level in such a manner that the higher the respiratory rate is, the higher is the fatigue level. The fatigue level calculation unit 212 may calculate fatigue levels in such a manner that a fatigue level corresponding to the time when a change amount in the respiratory rate exceeds the tolerated respiratory change amount is higher than a fatigue level corresponding to the time when a change amount in the respiratory rate does not exceed the tolerated respiratory change amount. The fatigue level calculation unit 212 may calculate a fatigue level in such a manner that the more different a change amount in the respiratory rate is from the tolerated respiratory change amount, the higher is the fatigue level. The fatigue level calculation unit 212 may calculate fatigue levels in such a manner that a fatigue level corresponding to the time when the respiration is irregular is higher than a fatigue level corresponding to the time when the respiration is not irregular. The fatigue level calculation unit 212 may calculate a fatigue level in such a manner that the more irregular the respiration is, the higher is the fatigue level.

In yet another example, the fatigue level calculation unit 212 may calculate a feature value related to perspiration, based on at least one of the patient image information and the patient biological information (in particular, information related to perspiration). The feature value related to the perspiration may include a perspiration amount. Subsequently, the fatigue level calculation unit 212 may determine whether or not a fatigue sign is exhibited in the perspiration, based on the feature value related to the perspiration. Examples of the fatigue sign exhibited in the perspiration may include: a sign indicating that the perspiration amount exceeds a tolerated perspiration amount. In that situation, the fatigue level calculation unit 212 may calculate fatigue levels in such a manner that a fatigue level corresponding to the time when the perspiration amount exceeds the tolerated perspiration amount is higher than a fatigue level corresponding to the time when the perspiration amount does not exceed the tolerated perspiration amount. The fatigue level calculation unit 212 may calculate a fatigue level in such a manner that the larger the perspiration amount is, the higher is the fatigue level.

In yet another example, the fatigue level calculation unit 212 may calculate a feature value related to the audio of the patient, based on the patient audio information. Subsequently, the fatigue level calculation unit 212 may determine whether or not a fatigue sign is exhibited in the audio of the patient, based on the feature value related to the audio of the patient. Examples of the fatigue sign exhibited in the audio may include: a sign indicating that the patient is uttering vocabulary expressing fatigue (e.g., at least one of the expression “tired”, the expression “painful”, and the expression “can't continue”). In that situation, the fatigue level calculation unit 212 may calculate fatigue levels in such a manner that a fatigue level corresponding to the time when the patient is uttering vocabulary expressing fatigue is higher than a fatigue level corresponding to the time when the patient is not uttering vocabulary expressing fatigue.

The fatigue level calculation unit 212 may calculate a fatigue level based on one of the plurality of types of feature values described above. The fatigue level calculation unit 212 may calculate a fatigue level based on a combination of at least two of the plurality of types of feature values described above. The fatigue level calculation unit 212 may determine whether or not the state of the patient exhibits one of the plurality of types of fatigue signs described above, so as to calculate a fatigue level based on the determined result. The fatigue level calculation unit 212 may determine whether or not the state of the patient exhibits a combination of at least two of the plurality of types of fatigue signs described above, so as to calculate a fatigue level based on the determined result.

The fatigue level calculation unit 212 may calculate fatigue levels that can be expressed with binary numbers. For example, the fatigue level calculation unit 212 may calculate fatigue levels in such a manner that a fatigue level corresponding to the time when the patient is fatigued is expressed with a first value (e.g., the value 1), whereas a fatigue level corresponding to the time when the patient is not fatigued is expressed with a second value (e.g., the value 0) different from the first value. More specifically, when a larger number of fatigue signs than a prescribed number are exhibited in the state of the patient, the fatigue level calculation unit 212 may determine that the patient is fatigued so as to calculate the fatigue level as the first value. On the contrary, when a larger number of fatigue signs than the prescribed number are not exhibited in the state of the patient, the fatigue level calculation unit 212 may determine that the patient is not fatigued so as to calculate the fatigue level as the second value.

In another example, the fatigue level calculation unit 212 may calculate fatigue levels that can be expressed with multiple values or continuous values. For example, the fatigue level calculation unit 212 may calculate a fatigue level in such a manner that the more fatigued the patient is, the larger value the fatigue level has. More specifically, for example, the fatigue level calculation unit 212 may calculate a fatigue level that varies in accordance with at least one of the quantity, the type, and the extent of fatigue signs exhibited in the state of the patient.

The fatigue level calculation unit 212 may calculate a fatigue level by using a fatigue level calculation engine that, in response to an input of at least one of the plurality of types of feature values related to the state of the patient, outputs the fatigue level of the patient. Alternatively, the fatigue level calculation unit 212 may calculate a fatigue level by using a fatigue level calculation engine that, in response to an input of the patient state information, outputs the fatigue level of the patient. The fatigue level calculation engine may include a rule-based fatigue level calculation engine. The fatigue level calculation engine may include a fatigue level calculation engine that can be trained. Examples of the fatigue level calculation engine that can be trained include an engine including a machine learning model that uses at least one of a neural network, a support vector machine, and a Bayesian network. The machine learning model in these examples may be trained by using a supervised learning method including a correct answer label, may be trained by using a clustering method, or may be trained by using reinforcement learning. It is desirable to have the fatigue level calculation engine generated in advance, before the rehabilitation assistance system SYS starts the rehabilitation assistance operation. For example, the rule-based fatigue level calculation engine may be generated by measuring a state of a patient who is actually doing exercise, identifying a fatigue level of the patient based on the patient's self-declaration or the assistant's judgment, and calculating a relationship between the measured state of the patient and the identified fatigue level (i.e., generating a rule for calculating a fatigue level from a state of the patient, based on the measured state of the patient and the identified fatigue level). For example, the rule-based fatigue level calculation engine may be generated based on a relationship between a state of a patient and a fatigue level that is theoretically derived or predicted from a simulation. For example, the fatigue level calculation engine that can be trained may be generated by measuring a state of a patient while the exercise is actually being performed, identifying a fatigue level of the patient based on the patient's self-declaration or the assistant's judgment, and updating a parameter of the fatigue level calculation engine so as to reduce (or minimize, preferably) the difference between an output of the fatigue level calculation engine obtained when the measured state of the patient is input to the fatigue level calculation engine and the identified fatigue level (i.e., a correct answer label).

After that, the FB information generation unit 213 generates the FB information based on the fatigue level calculated in step S22 (step S22).

In this situation, ideally, it is usually desirable when the actual fatigue level of the patient who is doing exercise for rehabilitation is equal to an ideal fatigue level corresponding to the load of the exercise. In other words, when the actual fatigue level of the patient is equal to the ideal fatigue level, it is assumed that the load of the exercise performed by the patient (i.e., the load imposed on the patient by the exercise) is an appropriate load with which advantageous effects of the rehabilitation can properly be enjoyed. On the contrary, when the actual fatigue level of the patient is higher than the ideal fatigue level, it is assumed that the load of the exercise performed by the patient is larger than the appropriate load. In that situation, because the burden on the patient is larger, there is a possibility that the patient may not be able to do the exercise safely. In other words, it is assumed that the load of the exercise performed by the patient and safety of the patient are not satisfactorily balanced. In contrast, when the actual fatigue level of the patient is lower than the ideal fatigue level, it is assumed that the load of the exercise performed by the patient is smaller than the appropriate load. In that situation, it is assumed that safety of the patient doing the exercise is excessively prioritized and, as a result, there is a possibility that the patient may not be able to sufficiently enjoy the advantageous effects of the rehabilitation. In other words, it is assumed that the load of the exercise performed by the patient and safety of the patient are not satisfactorily balanced.

As explained above, it is possible to use magnitude correlations between the fatigue level of the patient and the ideal fatigue level, for evaluating whether or not the load of the exercise being performed by the patient is appropriate. In other words, it is possible to use the magnitude correlations between the fatigue level of the patient and the ideal fatigue level for evaluating whether or not the load of the exercise being performed by the patient and safety of the patient are satisfactorily balanced. For this reason, the FB information generation unit 213 may generate the FB information based on a magnitude correlation between the fatigue level calculated in step S22 (i.e., the actual fatigue level) and a prescribed fatigue threshold value matching the ideal fatigue level or being determined based on the ideal fatigue level.

For example, when the fatigue level is equal to the fatigue threshold value, it is assumed that the load of the exercise is appropriate (i.e., the load of the exercise and safety of the patient are satisfactorily balanced). For this reason, in that situation, the FB information generation unit 213 may generate FB information including information that is desirable to be presented to at least one of the patient and the assistant in the situation where the load of the exercise is appropriate (i.e., the load of the exercise and safety of the patient are satisfactorily balanced). Alternatively, when the load of the exercise is appropriate, it is also assumed that the rehabilitation has no particular problem. Accordingly, when the load of the exercise is appropriate, the FB information generation unit 213 may omit generating the FB information.

For example, when the fatigue level exceeds the fatigue threshold value, it is assumed that the load of the exercise is not appropriate (i.e., the load of the exercise is larger than the appropriate load, so that the load of the exercise and safety of the patient are not satisfactorily balanced). Accordingly, in that situation, the FB information generation unit 213 may generate FB information including information that is desirable to be presented to at least one of the patient and the assistant in the situation where the load of the exercise is larger than the appropriate load (i.e., the load of the exercise and safety of the patient are not satisfactorily balanced).

For example, when the fatigue level is lower than the fatigue threshold value, it is assumed that the load of the exercise is not appropriate (i.e., the load of the exercise is smaller than the appropriate load, so that the load of the exercise and safety of the patient are not satisfactorily balanced). Accordingly, in that situation, the FB information generation unit 213 may generate FB information including information that is desirable to be presented to at least one of the patient and the assistant in the situation where the load of the exercise is smaller than the appropriate load (i.e., the load of the exercise and safety of the patient are not satisfactorily balanced).

The fatigue threshold value may be set based on the load of the exercise currently performed by the patient (i.e., the exercise presented to the patient as the exercise to be performed by the patient) and the fatigue level of the patient. The load of the exercise currently performed by the patient is determined according to a menu of the exercise performed by the patient. For this reason, the fatigue threshold value may be set based on the menu of the exercise currently performed by the patient and the fatigue level of the patient. For example, when the load of the exercise currently performed by the patient is appropriate, the fatigue level of the patient is equal to the ideal fatigue level. Accordingly, the fatigue threshold value may be set to the fatigue level of the patient that is expected on the assumption that the load of the exercise currently performed by the patient is appropriate.

The ideal fatigue level described above does not necessarily have to be a single value and may be a value within a certain range between a lower limit value and an upper limit value. For this reason, the FB information generation unit 213 may generate the FB information based on a magnitude correlation between the fatigue level and a prescribed upper limit fatigue threshold value matching an upper limit value of the ideal fatigue level or being determined based on the upper limit value of the ideal fatigue level and a magnitude correlation between the fatigue level and a prescribed lower limit fatigue threshold value matching a lower limit value of the ideal fatigue level or being determined based on the lower limit value of the ideal fatigue level. For example, when the fatigue level is lower than the upper limit fatigue threshold value and is higher than the lower limit fatigue threshold value, it is assumed that the load of the exercise is appropriate. Accordingly, in that situation, the FB information generation unit 213 may generate FB information including information that is desirable to be presented to at least one of the patient and the assistant in the situation where the load of the exercise is appropriate. For example, when the fatigue level exceeds the upper limit fatigue threshold value, it is assumed that the load of the exercise is larger than the appropriate load. Accordingly, in that situation, the FB information generation unit 213 may generate FB information including information that is desirable to be presented to at least one of the patient and the assistant in the situation where the load of the exercise is larger than the appropriate load. As another example, when the fatigue level is lower than the lower limit fatigue threshold value, it is assumed that the load of the exercise is smaller than the appropriate load. Accordingly, in that situation, the FB information generation unit 213 may generate FB information including information that is desirable to be presented to at least one of the patient and the assistant in the situation where the load of the exercise is smaller than the appropriate load. In these situations, the lower limit fatigue threshold value is typically smaller than the upper limit fatigue threshold value. However, one of the upper limit fatigue threshold value and the lower limit fatigue threshold value may be a threshold value that is set regardless of the other of the two threshold values, namely, the upper limit fatigue threshold value and the lower limit fatigue threshold value. Similarly to the fatigue threshold value, it is also acceptable to set the upper limit fatigue threshold value and the lower limit fatigue threshold value each based on the load of the exercise currently performed by the patient (i.e., the exercise presented to the patient as the exercise to be performed by the patient) and the fatigue level of the patient. For example, when the load of the exercise currently performed by the patient is appropriate, there is little possibility that the fatigue level of the patient may become high enough to exceed the certain range expected to be ideal fatigue levels. For this reason, the upper limit fatigue threshold value may be set to the upper limit value of the range of fatigue levels of the patient that are expected on the assumption that the load of the exercise currently performed by the patient is appropriate. As another example, when the load of the exercise currently performed by the patient is appropriate, there is little possibility that the fatigue level of the patient may become low enough to be lower than the certain range expected to be ideal fatigue levels. For this reason, the lower limit fatigue threshold value may be set to the lower limit value of the range of fatigue levels of the patient that are expected on the assumption that the load of the exercise currently performed by the patient is appropriate.

As explained above, the FB information may include the patient FB information to be presented to the patient. In that situation, the fatigue level calculation unit 212 may generate the patient FB information to be presented to a single patient based on the fatigue level of the single patient.

As explained above, the FB information may include the assistant FB information to be presented to the assistant. In that situation, the fatigue level calculation unit 212 may generate the assistant FB information to be presented to a single assistant based on the fatigue level of at least one patient assisted by the single assistant. For example, when a single assistant is assisting a single patient, the fatigue level calculation unit 212 may generate the assistant FB information to be presented to the assistant based on the fatigue level of the single patient. As another example, when a single assistant is assisting a plurality of patients, the fatigue level calculation unit 212 may generate the assistant FB information to be presented to the assistant based on fatigue levels of the plurality of patients.

When the patient FB information is generated in step S22, the FB information generation unit 213 transmits, by using the communication apparatus 23, the patient FB information generated in step S23 to one of the patient terminals 1 used by the one of the patients to whom the patient FB information should be presented (step S24). In other words, the FB information generation unit 213 outputs the patient FB information to the patient terminal 1. As a result, by using the communication apparatus 16, the output control unit 142 of the patient terminal 1 receives the patient FB information from the rehabilitation assistance server 2 (step S13). After that, the output control unit 142 controls the output apparatus 18 so as to present (i.e., output) the patient FB information received in step S13 to the patient (step S14). As a result, the patient is able to recognize the patient FB information.

The patient FB information may include exercise menu information related to a menu of the exercise to be performed by the patient. In that situation, the FB information generation unit 213 may generate patient FB information including the exercise menu information so that the load of the exercise and safety of the patient are satisfactorily balanced. For example, when the fatigue level exceeds a first fatigue threshold value or a first upper limit fatigue threshold value, it is assumed that the load of the exercise performed by the patient is larger than the appropriate load. In that situation, to maintain safety of the patient, it is desirable to make the load of the exercise to be performed by the patient smaller than the load of the exercise currently performed by the patient. Accordingly, when the fatigue level exceeds the first fatigue threshold value or the first upper limit fatigue threshold value, the FB information generation unit 213 may generate patient FB information including the exercise menu information indicating a menu of exercises having a load smaller than the load of the exercise currently performed by the patient. On the contrary, for example, when the fatigue level is lower than the first fatigue threshold value or a first lower limit fatigue threshold value, it is assumed that the load of the exercise performed by the patient is smaller than the appropriate load. In that situation, to enhance advantageous effects of the rehabilitation, it is desirable to make the load of the exercise to be performed by the patient larger than the load of the exercise currently performed by the patient. Accordingly, when the fatigue level is lower than the first fatigue threshold value or the first lower limit fatigue threshold value, the FB information generation unit 213 may generate patient FB information including the exercise menu information indicating a menu of exercises having a load larger than the load of the exercise currently performed by the patient.

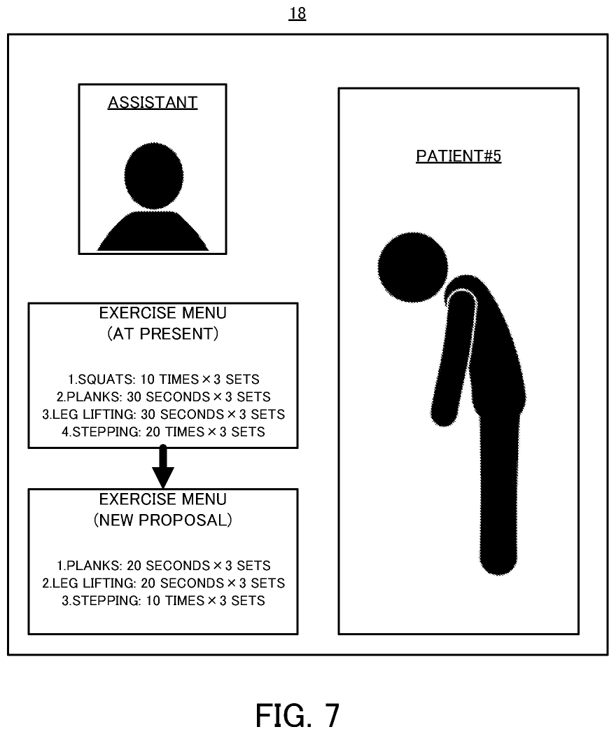

FIG. 7 shows an output example (a display example) of the patient FB information including the exercise menu information realized by the output apparatus 18. FIG. 7 shows the example in which the output apparatus 18 displays a menu of exercises to be performed by the patient (i.e., exercise menu information which is substantially represented by a menu of exercises newly proposed to the patient), together with a menu of exercises currently performed by the patient. In the example shown in FIG. 7, the menu of exercises indicated in the exercise menu information is different from the menu of exercises currently performed by the patient, for not including the squats and having a smaller number of times for the planks, the leg lifting, and the stepping. In other words, in the example shown in FIG. 7, the load of the exercise indicated by the exercise menu information is smaller than the load of the exercise currently performed by the patient. Thus, FIG. 7 shows the example of the exercise menu information generated in the situation where the fatigue level is determined to exceed the first fatigue threshold value or the first upper limit fatigue threshold value. As a result of the patient being presented with such exercise menu information, the patient is able to do the exercise having an appropriate load. Consequently, the patient is able to do the exercise safely without the occurrence of an accident such as a fall, while properly enjoying the advantageous effects of the rehabilitation. In other words, the patient is able to do the exercise while ensuring that the load of the exercise and safety are satisfactorily balanced.

The FB information generation unit 213 may generate exercise menu information indicating a plurality of mutually-different menus of exercises. In that situation, the patient is presented with the plurality of mutually-different menus of exercises. By using the input apparatus 17, the patient may select one of the plurality of mutually-different menus of exercises, as a menu of exercises to be performed by the patient. In that situation, the patient is able to do the exercises of his/her liking, while ensuring that the load of the exercise and safety are satisfactorily balanced.

Under the control of the FB information generation unit 213, the output apparatus 18 may present the patient with the exercise menu information, while the patient is doing exercise. In that situation, the menu of the exercises performed by the patient may be changed in a real-time manner in parallel with the exercise of the patient. In another example, the patient may perform an exercise for rehabilitation multiple times. In that situation, under the control of the FB information generation unit 213, the output apparatus 18 may present exercise menu information generated based on a fatigue level of the patient who has done the exercise for the N-th time (where N is an integer of 1 or larger), to the patient when the patient starts doing the exercise for the (N+1)-th time.

A default (i.e., initial) menu of exercises to be performed by the patient may be determined in advance by the assistant. Alternatively, the default menu of exercises to be performed by the patient may be determined in advance by an exercise menu determination engine that, in response to an input of attributes of the patient and a disease status of the patient or the like, outputs a menu of exercises to be performed by the patient. The exercise menu determination engine may include, for example, an exercise menu determination engine that can be trained (e.g., an engine including a machine learning model of a neural network or the like). In that situation, the exercise menu determination engine may be generated by updating a parameter of the exercise menu determination engine so as to reduce (or minimize, preferably) the difference between an output of the exercise menu determination engine obtained when the attributes of the patient and the disease state of the patient are input to the exercise menu determination engine and a menu of exercises actually proposed to the patient by the assistant (i.e., a correct answer label).

The patient FB information may include rest information related to proposing having a rest to the patient. In that situation, the FB information generation unit 213 may generate patient FB information including the rest information so that the load of the exercise and safety of the patient are satisfactorily balanced. For example, when the fatigue level exceeds a second fatigue threshold value or a second upper limit fatigue threshold value, it is assumed that the load of the exercise performed by the patient is larger than the appropriate load. In that situation, to maintain safety of the patient, the FB information generation unit 213 may generate patient FB information including the rest information proposing the patient to have a rest. In this situation, the second fatigue threshold value used for generating the rest information may be the same as or may be different from the abovementioned first fatigue threshold value used for generating the exercise menu information. Similarly, the second upper limit fatigue threshold value used for generating the rest information may be the same as or may be different from the abovementioned first upper limit fatigue threshold value used for generating the exercise menu information.

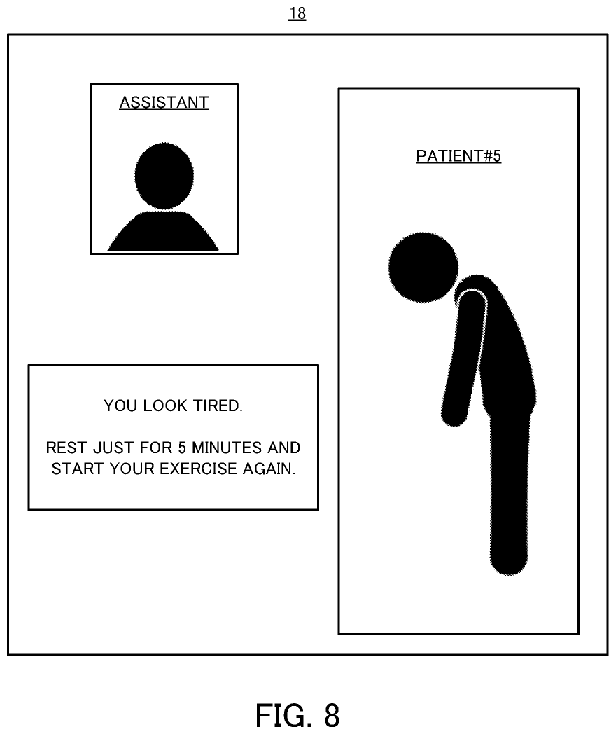

FIG. 8 shows an output example (a display example) of the patient FB information including the rest information realized by the output apparatus 18. As shown in FIG. 8, the rest information may include a text message suggesting having a rest to the patient. However, the rest information may include arbitrary information proposing the patient to have a rest. As a result the patient being presented with such rest information, the patient is able to do the exercise safely, while preventing the occurrence of an accident such as a fall that may be caused by excessive fatigue. In other words, the patient is able to do the exercise while ensuring that the load of the exercise and safety are satisfactorily balanced.

The patient FB information may include motivation increase information to increase motivation of the patient doing the exercise. In other words, the patient FB information may include the motivation increase information that includes arbitrary information for increasing the motivation of the patient doing the exercise. In that situation, the FB information generation unit 213 may generate patient FB information including the motivation increase information so that the load of the exercise and safety of the patient are satisfactorily balanced. For example, when the movements of the patient identified from the patient image information are slower than the movements that are naturally expected, although the fatigue level is lower than a third fatigue threshold value or a third lower limit fatigue threshold value, it is assumed that the patient is not properly doing the exercise although the patient has energy to spare. In that situation, the FB information generation unit 213 may generate patient FB information including the motivation increase information, so that the patient is able to have advantageous effects of the rehabilitation at a maximum level by doing the exercise properly. In this situation, the third fatigue threshold value used for generating the motivation increase information may be the same as or may be different from at least one of the abovementioned first fatigue threshold value used for generating the exercise menu information and the abovementioned second fatigue threshold value used for generating the rest information. Similarly, the third lower limit fatigue threshold value used for generating the motivation increase information may be the same as or may be different from the abovementioned first lower limit fatigue threshold value used for generating the exercise menu information. Further, the motivation increase information may include cheering information related to cheering up the patient doing the exercise. In other words, the motivation increase information may include arbitrary cheering information for cheering up the patient doing the exercise.

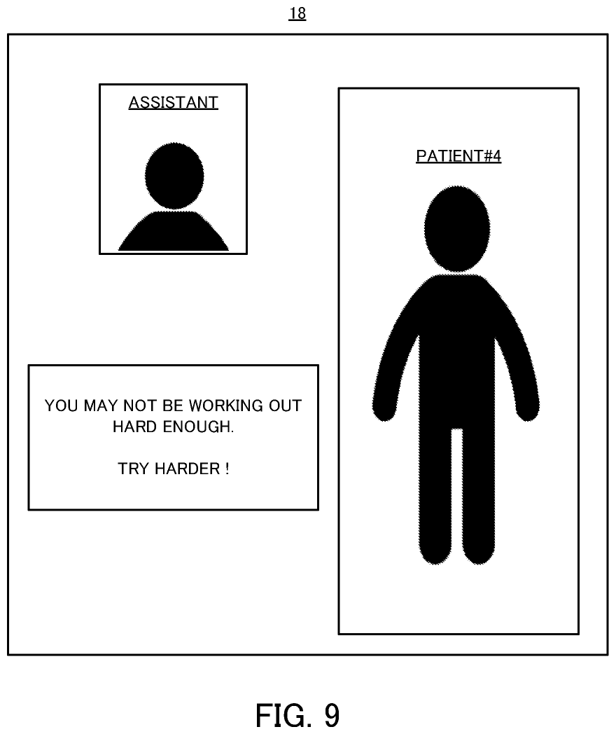

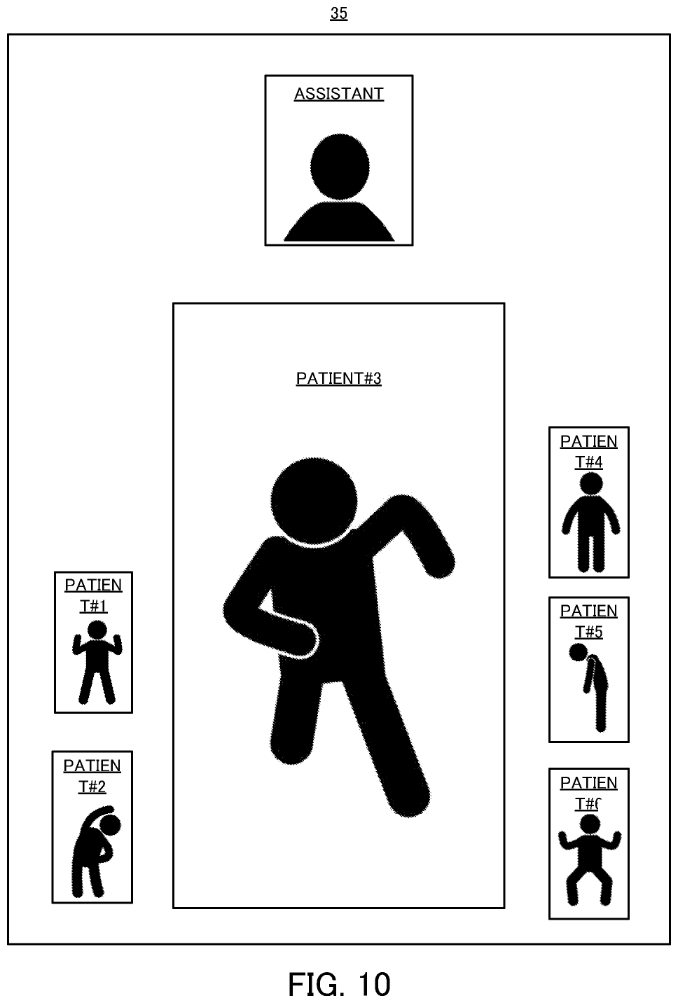

FIG. 9 shows an output example (a display example) of the patient FB information including the motivation increase information realized by the output apparatus 18. As shown in FIG. 9, the motivation increase information may include a text message for increasing motivation of the patient. However, the motivation increase information may include arbitrary information for increasing the motivation of the patient. For example, the motivation increase information may include an image in which a favorite character or person of the patient is cheering up the patient. As a result the patient being presented with such motivation increase information, the patient is able to do the exercise so as to properly enjoy advantageous effects of the rehabilitation. In other words, the patient is able to do the exercise while ensuring that the load of the exercise and safety are satisfactorily balanced.

Returning to the description of FIG. 6, when the assistant FB information is generated in step S22, the FB information generation unit 213 transmits, by using the communication apparatus 23, the assistant FB information generated in step S23 to the assistant terminal 3 used by the single assistant to whom the assistant FB information is to be presented (step S24). In other words, the FB information generation unit 213 outputs the assistant FB information to the assistant terminal 3. As a result, by using the communication apparatus 33, the output control unit 311 of the assistant terminal 3 receives the assistant FB information from the rehabilitation assistance server 2 (step S31). After that, the output control unit 311 controls the output apparatus 35 so as to present (i.e., output) the assistant FB information received in step S31 to the assistant (step S32). As a result, the assistant is able to recognize the assistant FB information.

The assistant FB information may include specific patient information related to a patient whose fatigue level satisfies a prescribed condition. In other words, the assistant FB information may include the specific patient information indicating whether or not the fatigue levels of the patients satisfy the prescribed condition. The prescribed condition may include a condition where the fatigue level exceeds a fourth fatigue threshold value or a fourth upper limit fatigue threshold value. In that situation, the specific patient information corresponds to information related to the patient having a high fatigue level (i.e., the patient who is fatigued). Alternatively, the prescribed condition may include a condition where the fatigue levels are lower than the fourth fatigue threshold value or a fourth lower limit fatigue threshold value. In that situation, the specific patient information corresponds to information related to patients having low fatigue levels (i.e., the patients who are not so fatigued).

In this situation, the fourth fatigue threshold value used for generating the specific patient information may be the same as or may be different from at least one of the abovementioned first to third fatigue threshold values used for generating the patient FB information. The fourth upper limit fatigue threshold value used for generating the specific patient information may be the same as or may be different from at least one of the abovementioned first and second upper limit fatigue threshold values used for generating the patient FB information. Further, the fourth lower limit fatigue threshold value used for generating the specific patient information may be the same as or may be different from at least one of the abovementioned first and third lower limit fatigue threshold values used for generating the patient FB information.

FIG. 10 shows an output example (a display example) of the assistant FB information including the specific patient information realized by the output apparatus 35. FIG. 10 shows the example in which the output apparatus 35 displays images showing patients, while the output mode of the image of a patient (hereinafter, “specific patient”; patient #3 in the example shown in FIG. 10) whose fatigue level satisfies a prescribed condition is arranged to be different from the output mode of the images of patients (hereinafter, “non-specific patients” patients #1, #2, and #4 to #6 in the example shown in FIG. 10) whose fatigue levels do not satisfy the prescribed condition. Typically, the output control unit 311 may output the assistant FB information so that the image of the specific patient is more conspicuous than the images of the non-specific patients. In this situation, the output mode of each image may include at least one of the size of the image, the thickness of an outer frame of the image, and the color of the outer frame of the image. FIG. 10 shows the example in which the size of the image of the specific patient is different from the size of the images of the non-specific patients.

As shown in FIG. 10, outputting (displaying) the assistant FB information including the specific patient information may be regarded to be equivalent to outputting (displaying) image information of the patients corresponding to whether or not the fatigue levels satisfy the prescribed condition. In that situation, the FB information generation unit 213 may generate image information representing images of the patients corresponding to whether or not the fatigue levels satisfy the prescribed condition, as the specific patient information. For example, when the images showing the patients are displayed so that the output mode of the image of the specific patient is different from the output mode of the images of the non-specific patients, the FB information generation unit 213 may generate, as the specific patient information, image information representing the image of the specific patient and image information representing the images of the specific patient to be output in an output mode different from that of the image information of the specific patient.

FIG. 10 shows the example in which the output apparatus 35 displays the images of the plurality of patients (i.e., the example in which the assistant is assisting the plurality of patients). However, even in the situation when the assistant is assisting a single patient, the output apparatus may display an image of the patient, while the output mode of images of specific patients is arranged to be different from the output mode of images of non-specific patients. In other words, the output apparatus 35 may display the image of the patient, while the output mode of the image of the patient used when the patient is a specific patient is different from the output mode of the image of the patient used when the patient is not a specific patient (i.e., is a non-specific patient).

In that situation, the output control unit 311 may control the output apparatus 35 so as to select the image of the specific patient based on the specific patient information and to arrange the output mode of the selected image to be different from the output mode of unselected images. In other words, the output control unit 311 may control the output apparatus 35 so that the output mode of the image of the specific patient indicated by the specific patient information is arranged to be different from the output mode of the images of the non-specific patients who are not indicated by the specific patient information. In that situation, the FB information generation unit 213 may be regarded to control the output apparatus 35 so as to arrange the output mode of the image of the specific patient to be different from the output mode of the images of the non-specific patients by using the specific patient information. Alternatively, when the specific patient information includes the image information of the patient as explained above, the output control unit 311 may control the output apparatus 35 so as to display the image represented by the image information included in the specific patient information.

When the output mode of the image of the specific patient is different from the output mode of the images of the non-specific patients, the assistant is able to identify the specific patient based on the output modes of the images. Accordingly, in the present example embodiment, the operation of displaying the images of the plurality of patients while arranging the output mode of the image of the specific patient to be different from the output mode of the images of the non-specific patients may be regarded to be substantially equivalent to an operation of presenting (informing, in the present example) the assistant with (of) the specific patient information indicating the specific patient. When the assistant is presented with the specific patient information in this manner, the assistant is able to determine whether or not the patients assisted by the assistant include one or more specific patients. As a result, the assistant is able to pay attention to the specific patient and to assist the specific patient while giving higher priority than the priority given to the non-specific patients. Consequently, even when the assistant is assisting the plurality of patients, the assistant is able to properly assist the patient that needs attention (i.e., the patient estimated to be in need of assistance) among the plurality of patients, without overlooking such a patient. Further, even when the assistant is assisting a single patient, the assistant is able to easily determine whether or not the single patient is in need of assistance and, as a result, it is possible to properly assist the patient.

In an example, when the assistant is presented with the specific patient information related to a patient whose fatigue level is high (i.e., a fatigued patient), the assistant is able to pay attention to the patient having the high fatigue level. In that situation, the assistant may provide the patient having the high fatigue level with advice suitable for the fact that the fatigue level is high. For example, the assistant may provide the patient having the high fatigue level with advice suggesting to have a rest. In other words, the assistant may provide the advice for the purpose of satisfactorily balancing the load of the exercise with safety of the patient. As a result, the patient is able to do the exercise safely, while preventing the occurrence of an accident such as a fall that may be caused by excessive fatigue. In other words, the patient is able to do the exercise, while ensuring that the load of the exercise and safety are satisfactorily balanced.

In another example, when the assistant is presented with the specific patient information related to a patient whose fatigue level is low (i.e., a patient who is not so fatigued), the assistant is able to pay attention to the patient having the low fatigue level. In that situation, the assistant may provide the patient having the low fatigue level with advice suitable for the fact that the fatigue level is low. For example, the assistant may provide the patient having the low fatigue level with advice prompting the patient to do exercise (i.e., cheering up the patient) so that the patient is able to have advantageous effects of the rehabilitation at a maximum level by properly doing the exercise. In other words, the assistant may provide the advice for the purpose of satisfactorily balancing the load of the exercise with safety of the patient. As a result, the patient is able to do the exercise so as to be able to properly enjoy the advantageous effects of the rehabilitation. In other words, the patient is able to do the exercise, while ensuring that the load of the exercise and safety are satisfactorily balanced.

In yet another example, the output control unit 311 may control the output apparatus 35 so as to display, without any modification, the plurality of images respectively showing the plurality of patients including the specific patient and the non-specific patients. For example, the output control unit 311 may control the output apparatus 35 so that the output mode of the image of the specific patient is the same as the output mode of the images of the non-specific patients. In that situation, however, the output control unit 311 may control the output apparatus 35 so as to change the display order of the images of the plurality of patients on the display screen of the output apparatus 35 based on the fatigue levels. For example, the output control unit 311 may control the output apparatus 35 so that the images of the plurality of patients are displayed in descending order of the fatigue levels, along a first section (e.g., an upper left section) toward a second section (e.g., a lower right section) on the display screen of the output apparatus 35.

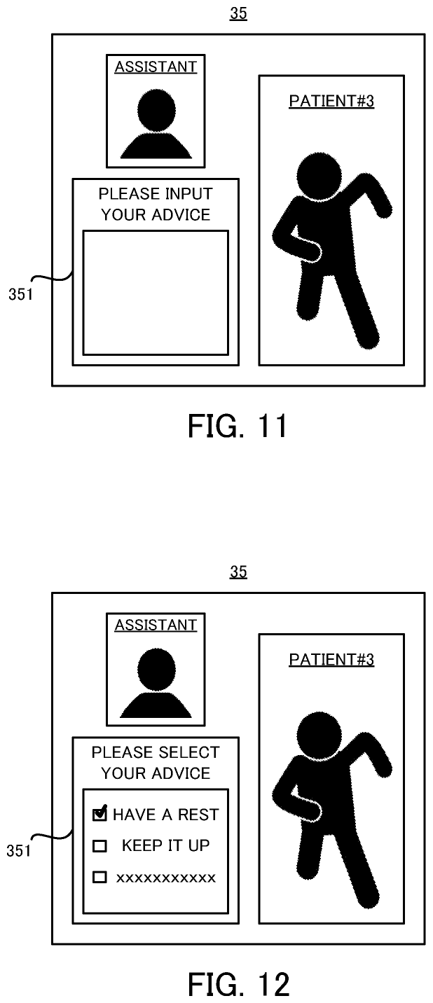

The output control unit 311 may control the output apparatus 35 so as to output (display in the present example) a user interface 351 used by the assistant to input advice for the patient. FIG. 11 shows an example of the user interface 351. By using the input apparatus 34, the assistant may input the advice for the patient on the user interface 351. For example, when the input apparatus 34 includes an operation apparatus such as a keyboard, the assistant may input the advice for the patient by operating the operation apparatus. As another example, when the input apparatus 34 includes an audio input apparatus such as a microphone, the assistant may input audio including the advice for the patient to the audio input apparatus. Advice information related to the advice input by the assistant via the user interface 351 may be presented to the patient as at least a part of the patient FB information that is desirable to be presented to the patient. More specifically, the communication apparatus 33 of the assistant terminal 3 may transmit the advice information to the rehabilitation assistance server 2, via the communication network 4. The communication apparatus 23 of the rehabilitation assistance server 2 may receive the advice information. The FB information generation unit 213 may generate patient FB information including the received advice information. The output control unit 142 of a relevant one of the patient terminals 1 may control the output apparatus 18, so as to output the advice information generated by the FB information generation unit 213 (e.g., so as to display a text message representing the advice information or to output audio representing the advice information). As a result, the advice information is presented to the patient. In other words, the assistant is able to provide the patient with the advice relatively easily, via the user interface 351.

When the output apparatus 35 outputs (displays) the user interface 351, the FB information generation unit 213 may generate, as assistant FB information, advice input screen information representing the user interface 351 (i.e., the input screen used by the assistant to input the advice for the patient). In that situation, the output control unit 311 may control the output apparatus 35 so as to display the user interface 351 based on the advice input screen information.

As shown in FIG. 12, the user interface 351 may include a plurality of mutually-different advice candidates. By using the input apparatus 34, the assistant may select at least one of the plurality of mutually-different advice candidates. Advice information related to at least one piece of advice selected by the user may be presented to the patient, similarly to the advice information related to the advice input by the assistant. As a result, it is possible to reduce trouble of the assistant in inputting the advice.

Based on the specific patient information, when the patients assisted by the assistant include a specific patient, the output control unit 311 may control the output apparatus 35 so as to automatically display the user interface 351. Alternatively, the output control unit 311 may control the output apparatus 35 so as to display the user interface 351 with desired timing. For example, the output control unit 311 may control the output apparatus 35 so as to display the user interface 351 when the assistant requests, via the input apparatus 34, that the user interface 351 be displayed.

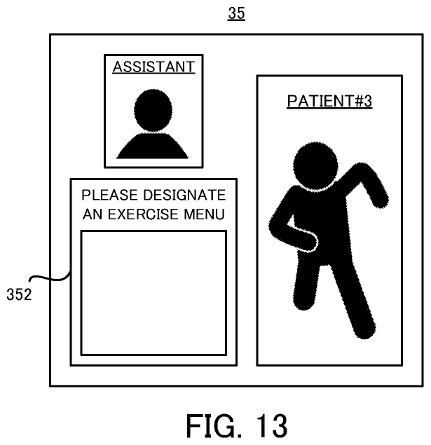

The output control unit 311 may control the output apparatus 35 so as to output (display, in the present example) a user interface 352 used by the assistant to designate a menu of exercises to be presented to a patient. FIG. 13 shows an example of the user interface 352. By using the input apparatus 34, the assistant may designate the menu of exercises to be presented to the patient on the user interface 352. For example, when the input apparatus 34 includes an operation apparatus such as a keyboard, the assistant may designate the menu of exercises to be presented to the patient, by operating the operation apparatus. As another example, when the input apparatus 34 includes an audio input apparatus such as a microphone, the assistant may input audio designating the menu of exercises to be presented to the patient, to the audio input apparatus. The menu of exercises designated by the assistant via the user interface 352 may be presented to the patient, as the exercise menu information explained above. More specifically, the communication apparatus 33 of the assistant terminal 3 may transmit the exercise menu information to the rehabilitation assistance server 2 via the communication network 4. The communication apparatus 23 of the rehabilitation assistance server 2 may receive the exercise menu information. The FB information generation unit 213 may generate patient FB information including the received exercise menu information. As a result, the exercise menu information is presented to the patient. In other words, the assistant is able to relatively easily present the patient with the menu of exercises desirable to be performed by the patient, via the user interface 352.

The assistant FB information may include fatigue level information related to the fatigue levels calculated by the fatigue level calculation unit 212. In that situation, based on the fatigue level information, the output control unit 311 may display images of the patients by using an output mode (i.e., a display mode) in which the images of the patients are kept in association with the fatigue levels. As a result, the assistant is able to recognize the fatigue levels of the patients. In another example, based on the fatigue level information, the output control unit 311 may configure output modes of the images of the patients to be output modes determined in accordance with the fatigue levels indicated by the fatigue level information. For example, the output control unit 311 may configure the colors of the outer frames of the images of the patients to be colors determined in accordance with the fatigue levels. More specifically, the output control unit 311 may configure the color of the outer frame of an image of a patient whose fatigue level falls within a first numerical value range to be a first color (e.g., blue), may configure the color of the outer frame of an image of another patient whose fatigue level falls within a second numerical value range larger than the first numerical value range to be a second color (e.g., yellow), and may configure the color of the outer frame of an image of yet another patient whose fatigue level falls within a third numerical value range larger than the second numerical value range to be a third color (e.g., red). In that situation, the assistant is able to recognize the fatigue levels of the patients.

### (3) TECHNICAL ADVANTAGEOUS EFFECTS OF THE REHABILITATION ASSISTANCE SYSTEM SYS

As explained above, the rehabilitation assistance server 2 calculates the fatigue levels of the patients and generates the FB information (i.e., at least one of the patient FB information and the assistant FB information) based on the fatigue levels. Accordingly, by using the patient FB information, the patients are able to do the exercise while ensuring that the load of the exercise and safety are satisfactorily balanced. Further, by using the assistant FB information, the assistant is able to assist the patients so that the patients are able to do the exercise while ensuring that the load of the exercise and safety are satisfactorily balanced. In this manner, by generating the FB information based on the fatigue levels, the rehabilitation assistance server 2 is able to assist the rehabilitation in such a manner that the load of the exercise performed by the patients for the rehabilitation and safety of the patients are satisfactorily balanced.

In particular, in the present example embodiment, even when a subject is doing the exercise in a place that is out of sight of the assistant (i.e., in a place to which the assistant is not able to pay sufficient attention), the rehabilitation assistance system SYS is able to assist the rehabilitation in such a manner that the load of the exercise performed by the patient for rehabilitation and safety of the patient are satisfactorily balanced. The reason is that, as explained above, the patient FB information is presented to the patient and/or the assistant FB information is presented to the assistant. More specifically, for example, even when the assistant is not able to assist the rehabilitation of a patient in person (e.g., when the assistant is remotely assisting the rehabilitation of the patient), the rehabilitation assistance system SYS is able to assist the rehabilitation in such a manner that the load of the exercise performed by the patient for rehabilitation and safety of the patient are satisfactorily balanced. For example, even when the assistant is in a rehabilitation institution, while a patient is at his/her residence or in another rehabilitation institution, the rehabilitation assistance system SYS is able to assist the rehabilitation in such a manner that the load of the exercise performed by the patient for rehabilitation and safety of the patient are satisfactorily balanced.

As explained above, when a subject is able to do the exercise while being out of sight of the assistant, the assistant (or the rehabilitation institution) is able to enjoy an advantageous effect where it is possible to efficiently assist the rehabilitation of the patient. In particular, when a single assistant assists a plurality of patients, the assistant (or the rehabilitation institution) is able to enjoy an advantageous effect where it is possible to efficiently assist rehabilitation of the plurality of patients. The patients are also able to enjoy an advantageous effect where it is possible to have the assistance from a rehabilitation institution that is difficult to visit (e.g., a rehabilitation institution in an area distant from the patient's residence). Furthermore, the patients are able to enjoy an advantageous effect where it is possible to save the trouble of making a visit to the institution for the rehabilitation.

Further, in the present example embodiment, even when a subject is doing exercise within sight of the assistant (i.e., in a place to which the assistant is able to pay sufficient attention), the rehabilitation assistance system SYS is able to assist the rehabilitation in such a manner that the load of the exercise performed by the patient for rehabilitation and safety of the patient are satisfactorily balanced. The reasons is that, as explained above, the situation is the same where the patient FB information is presented to the patient and/or the assistant FB information is presented to the assistant. In addition, when a single assistant assists a plurality of patients, it is possible to enjoy an advantageous effect where the burden imposed on the assistant when assisting the rehabilitation of the plurality of patients is reduced. The reason is that, as explained above, the assistant is presented with the assistant FB information generated on the assumption that the single assistant assists the plurality of patients.

### (4) MODIFICATIONS

### (4-1) First Modification

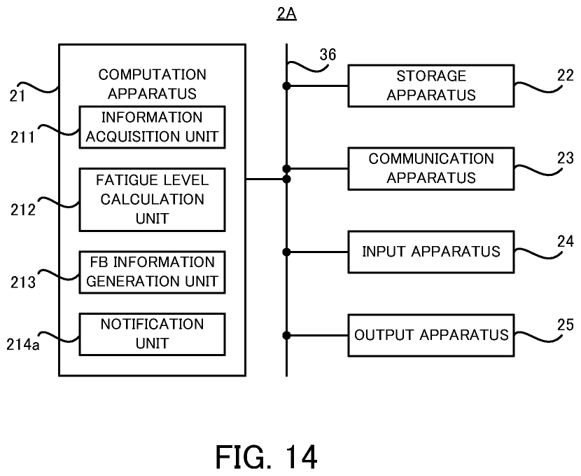

Next, a rehabilitation assistance server 2a in a first modification will be explained, with reference to FIG. 14. FIG. 14 is a block diagram illustrating a configuration of the rehabilitation assistance server 2a in the first modification.

As shown in FIG. 14, the rehabilitation assistance server 2a in the first modification is different from the rehabilitation assistance server 2 described above in that the computation apparatus 21 includes a notification unit 214a, which is a specific example of the “notification unit”. The rest of the configuration of the rehabilitation assistance server 2a may be the same as the rest of the configuration of the rehabilitation assistance server 2.

The notification unit 214a determines whether or not each patient is in an abnormal state, based on the patient state information. Examples of the abnormal state may include a state in which the patient doing exercise for rehabilitation is in a state different from a normal state. Examples of the abnormal state may include at least one of: a state in which an accident has occurred (e.g., a state in which the patient fell); and a state in which there is a possibility that an accident may occur (e.g., a state in which the patient has a possibility of falling).

Further, when it is determined that the patient is in an abnormal state, the notification unit 214a notifies a prescribed notification destination that the patient is in an abnormal state. Examples of the prescribed notification destination may include at least one of an institution and a person that is desirable to be notified that the patient is in an abnormal state. Examples of the prescribed notification destination may include an information processing apparatus that is usable by at least one of the institution and the person that is desirable to be notified that the patient is in an abnormal state. For instance, examples of the prescribed notification destination may include a fire station capable of arranging an ambulance. For instance, examples of the prescribed notification destination may include a medical institution or a rehabilitation institution which the patient regularly visits. For instance, examples of the prescribed notification destination may include a medical provider (e.g., a doctor) who is in charge of diagnosing and/or treating the patient. For instance, examples of the prescribed notification destination may include the assistant.

In the first modification configured as described above, when the patient is in an abnormal state, the prescribed notification destination is notified of the situation. Accordingly, even when the patient goes into an abnormal state while doing the exercise for rehabilitation in a place out of sight of the assistant, the prescribed notification destination is able to promptly take measures to assist the patient in the abnormal state. Consequently, it is possible to more properly maintain safety of the patient who is doing the exercise for rehabilitation in the place that is out of sight of the assistant.

### (4-2) Second Modification

As explained above, the output apparatus 35 of the assistant terminal 3 may display the images of the patients. In the second modification, the output apparatus 35 may display (output) the images of the patients in an output mode (a display mode in the present example) that protects privacy of the patients, under control of the rehabilitation assistance server 2. For example, the output apparatus 35 may display the images of the patients by using an output mode in which the patients' faces are not recognizable by the assistant. In an example, the output apparatus 35 may display the images of the patients by using an output mode in which the patients faces are blurred with a mosaic pattern. For example, the output apparatus 35 may display the images of the patients by using an output mode in which the body shapes of the patients are not recognizable by the assistant. In an example, the output apparatus 35 may display the images of the patients by using an output mode in which the body shapes of the patients are the same no matter which patient an image is displayed of. For example, the output apparatus 35 may display the images of the patients by using an output mode in which the bodies of the patients are expressed with a wire model (a so-called model that schematically expresses the skeleton of each patient). For example, the output apparatus 35 may display the images of the patients by using an output mode in which the backgrounds of the patients (e.g., the room at the residence of each patient) are not recognizable by the assistant. In an example, the output apparatus 35 may display the images of the patients by using an output mode in which the waveforms of the patients are blurred with a mosaic pattern or the backgrounds of the patients are overwritten with a prescribed background. In these situations, the output apparatus 35 may use an output mode that protects privacy of the patients to an extent that the assistant is able to understand the state of each patient.

In the second modification configured as described above, it is possible to properly protect privacy of the patients whose images are displayed on the assistant terminal 3. Accordingly, even in the situation where an assistant who is assisting a patient by using the assistant terminal 3 is unfamiliar to the patient (or is an assistant different from the assistant who usually assists the patient; the same applies hereinafter), the patient does not need to worry about his/her personal information being disclosed to the assistant who is unfamiliar to the patient. In other words, the patient is able to do the exercise for rehabilitation, not only when the assistant who usually assists the patient is assisting the patient by using the assistant terminal 3, but also when the assistant who is unfamiliar to the patient is assisting the patient by using the assistant terminal 3. Consequently, it is possible to provide the patient with relatively more opportunities to do exercise. Further, even when the assistant who usually assists the patient is unable to assist the patient, the other assistant who is unfamiliar to the patient is able to assist the patient while protecting privacy of the patient. As a result, there is more flexibility in selecting an assistant who is to assist the patient.

### (4-3) Third Modification

In the above description, the FB information generation unit 213 generates the FB information based on the fatigue levels of the patients. However, in a third modification, the FB information generation unit 213 may generate FB information based on information different from the fatigue levels of the patients.

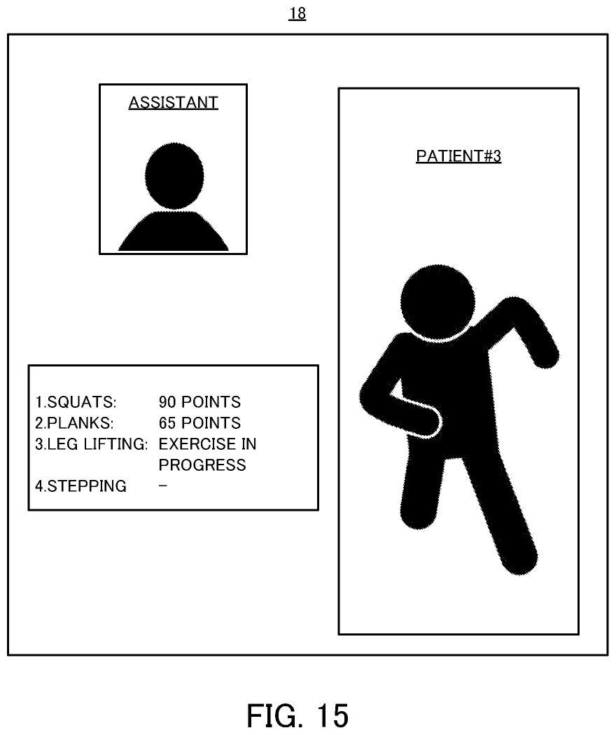

For example, the FB information generation unit 213 may generate patient FB information including score information related to a score of the exercise that was performed by a patient. To generate the score information, the FB information generation unit 213 may calculate a feature value related to movements of the patient, based on at least one of the patient image information and the patient biological information (in particular, information related to the movements of the patient). After that, based on the feature value related to the movements of the patient, the FB information generation unit 213 may generate the score information in such a manner that the larger the difference is between the movements of the patient and the movements that are naturally expected, the smaller is the score. FIG. 15 shows an output example (a display example) of the patient FB information including the score information realized by the output apparatus 18. As shown in FIG. 15, the score information may include numerical values that quantitatively express scores. However, the score information may include arbitrary information indicating scores. As a result of the patient being presented with such score information, the patient is able to objectively understand the quality of the exercise that was performed by the patient. As a result, the patient is able to do the exercise so as to increase the score (i.e., so as to properly enjoy advantageous effects of the rehabilitation). In other words, the patient is able to do the exercise while ensuring that the load of the exercise and safety are satisfactorily balanced.

For example, the FB information generation unit 213 may generate patient FB information including response information indicating a response to a request of a patient. To generate the response information, the FB information generation unit 213 may identify the request of the patient based on the patient state information. For example, the FB information generation unit 213 may identify the request of the patient based on the patient audio information. After that, the FB information generation unit 213 may generate the response information indicating a response granting the request of the patient. In an example, when the patient uttered speech such as “I want to rest” or “I'm tired”, the FB information generation unit 213 may identify, based on the patient audio information, that the request of the patient is a request for having a rest. In that situation, the FB information generation unit 213 may generate response information corresponding to the abovementioned rest information, so that the patient is able to have a rest. In another example, when the patient uttered speech such as “The exercise this time was painful”, the FB information generation unit 213 may identify, based on the patient audio information, that the request of the patient is a request for reducing the load of the exercise. In that situation, in order to propose a new menu of exercises having a smaller load to the patient, the FB information generation unit 213 may generate response information corresponding to the abovementioned exercise menu information.

For example, the FB information generation unit 213 may generate assistant FB information including patient request information related to a request of a patient. To generate the patient request information, the FB information generation unit 213 may identify the request of the patient based on the patient state information, similarly to the example of generating the response information. On such occasion, the FB information generation unit 213 may prompt the patient to input a request by using the output apparatus 18, so that the patient is able to input the request of the patient via at least one of the microphone 12 and the input apparatus 17. For example, when the output apparatus 18 includes a display apparatus, the FB information generation unit 213 may control the output apparatus 18 so as to display a text message reading “Please input your request”. For example, when the output apparatus 18 includes an audio output apparatus (a speaker), the FB information generation unit 213 may control the output apparatus 18 so as to output audio saying “Please input your request”. As a result, the FB information generation unit 213 is able to generate the patient request information. When the assistant is presented with such patient request information, the assistant is able to recognize the request of the patient. As a result, the assistant is able to properly assist the patient in accordance with the request of the patient.

For example, the FB information generation unit 213 may generate assistant FB information including patient opinion information related to an opinion (e.g., an impression) of a patient including a request of the patient. To generate the patient opinion information, the FB information generation unit 213 may identify the opinion of the patient, based on the patient state information, similarly to the example of generating the response information or the patient request information. On such occasion, the FB information generation unit 213 may prompt the patient to input the opinion by using the output apparatus 18, so that the patient is able to input the opinion of the patient via at least one of the microphone 12 and the input apparatus 17. For example, when the output apparatus 18 includes a display apparatus, the FB information generation unit 213 may control the output apparatus 18 so as to display a text message reading “Please input your impression on today's exercise”. For example, when the output apparatus 18 includes an audio output apparatus (a speaker), the FB information generation unit 213 may control the output apparatus 18 so as to output audio saying “Please enter your impression on today's exercise”. As a result, the FB information generation unit 213 is able to generate the patient opinion information. When the assistant is presented with such patient request information, the assistant is able to recognize the request of the patient. As a result, the assistant is able to properly assist the patient in accordance with the request of the patient.

For instance, in the above description, the patient whose fatigue level satisfies the prescribed condition is referred to as the specific patient. However, it is also acceptable to refer to a patient who is in a prescribed state, regardless of the fatigue level, as a specific patient. In that situation, the FB information generation unit 213 may generate assistant FB information including specific patient information related to the patient who is in the prescribed state. To generate the specific patient information, the FB information generation unit 213 may extract the patient who is in the prescribed state, based on the patient state information. For example, based on the patient image information, the FB information generation unit 213 may extract a patient having a prescribed movement as the specific patient. In another example, based on the patient image information, the FB information generation unit 213 may extract a patient who is making prescribed audio as the specific patient. As yet another example, based on the patient biological information, the FB information generation unit 213 may extract a patient whose vital sign indicated in the patient biological information has a prescribed value, as the specific patient. In these situations, the rehabilitation assistance server 2 is able to present (inform) the assistant with (of) the patient to whom the assistant should pay attention, on account of not only the fatigue level, but various factors.

The output control unit 142 of any of the patient terminals 1 may control the output apparatus 18 so as to output information described below, in addition to the patient FB information. For example, as explained above, the output control unit 142 may control the output apparatus 18 so as to display an image of at least one of the patient and the assistant. When the single assistant is assisting a plurality of patients, the output control unit 142 of the patient terminal 1 used by one of the plurality of patients may control the output apparatus 18 so as to output at least one of an image and audio of the assistant in a time period during which the assistant is taking some kind of action (e.g., giving advice) for the one of the patients. In contrast, the output control unit 142 of the patient terminal 1 used by the one of the patients may control the output apparatus 18 so as not to output at least one of an image and audio of the assistant in a time period during which the assistant is not taking some kind of action for the one of the patients. The output control unit 142 may control the output apparatus 18 so as to display an image indicating specifics of the exercise presented to the patient (typically, a video showing an example of the exercise). The output control unit 142 may control the output apparatus 18 so as to output audio of the patient (e.g., speech saying “It's hard”, speech saying “I'm tired”, or speech saying “not yet”).

The output control unit 311 of the assistant terminal 3 may control the output apparatus 35 so as to output information described below, in addition to the assistant FB information. For example, as explained above, the output control unit 311 may control the output apparatus 35 so as to display an image of at least one of the patients and the assistant. The output control unit 311 may control the output apparatus 35 so as to output the advice information related to advice from the assistant for any of the patients (e.g., to display a text message indicating the advice information or to output audio indicating the advice information). When the single assistant is assisting a plurality of patients, the output control unit 311 may control the output apparatus 35 so as to output at least one of an image and audio of one of the patients for whom the assistant is taking some kind of action (e.g., giving advice). In contrast, the output control unit 311 may control the output apparatus 35 so as not to output at least one of an image and audio of one of the patients for whom the assistant is not taking some kind of action. The output control unit 311 may control the output apparatus 35 so as to display an image indicating specifics of the exercise presented to the patient (typically, a video indicating an example of the exercise). The output control unit 311 may control the output apparatus 35 so as to output audio of the patient (e.g., speech saying “It's hard”, speech saying “I'm tired”, or speech saying “not yet”).

### (4-4) Other Modifications

In the above description, the rehabilitation assistance server 2 is a different apparatus from the assistant terminal 3. However, it is also acceptable to incorporate at least a part of the logical processing blocks included in the computation apparatus 21 of the rehabilitation assistance server 2 into the assistant terminal 3. More specifically, at least one of the information acquisition unit 211, the fatigue level calculation unit 212, and the FB information generation unit 213 may be realized within the computation apparatus 31 of the assistant terminal 3. Alternatively, the rehabilitation assistance system SYS may include an apparatus in which the rehabilitation assistance server 2 and the assistant terminal 3 are integrally formed, instead of including the rehabilitation assistance server 2 and the assistant terminal 3 that are separate from each other.

In the above description, the rehabilitation assistance server 2 is a different apparatus from the patient terminals 1. However, it is also acceptable to incorporate at least a part of the logical processing blocks included in the computation apparatus 21 of the rehabilitation assistance server 2 into any of the patient terminals 1. More specifically, at least one of the information acquisition unit 211, the fatigue level calculation unit 212, and the FB information generation unit 213 may be realized within the computation apparatus 14 of any of the patient terminals 1. Alternatively, the rehabilitation assistance system SYS may include an apparatus in which the rehabilitation assistance server 2 and the patient terminals 1 are integrally formed, instead of including the rehabilitation assistance server 2 and the patient terminals 1 that are separated from each other.

The image of the patient generated by the camera 11 may be stored in the storage apparatus 15. In that situation, the image of the patient stored in the storage apparatus 15 may be used as an evidence indicating how the patient was doing the exercise for rehabilitation. Further, the images of the patient stored in the storage apparatus 15 may be used as an evidence indicating how the assistant was assisting the patient. However, to protect privacy of the patient, the image of the patient stored in the storage apparatus 15 may be discarded with prescribed timing. For example, the image of the patient stored in the storage apparatus 15 may be discarded when a certain length of time (e.g., one day or one week) has elapsed since the image of the patient was generated. For example, the image of the patient stored in the storage apparatus 15 may be discarded when the calculation of the fatigue level (or the calculation of the feature value used for calculating the fatigue level) based on the image is completed.

The audio of the patient generated by the microphone 12 may be stored in the storage apparatus 15. In that situation, the audio of the patient stored in the storage apparatus 15 may be used as an evidence indicating how the patient was doing the exercise for rehabilitation. Further, the audio of the patient stored in the storage apparatus 15 may be used as an evidence indicating how the assistant was assisting the patient. However, to protect privacy of the patient, the audio of the patient stored in the storage apparatus 15 may be discarded with prescribed timing. In that situation, the timing with which the audio of the patient is discarded may be the same as or may be different from the timing with which the image of the patient is discarded.

The patient biological information generated by the biological sensor 13 may be stored in the storage apparatus 15. In that situation, the patient biological information of the patient stored in the storage apparatus 15 may be used as an evidence indicating how the patient was doing the exercise for rehabilitation. Further, the patient biological information of the patient stored in the storage apparatus 15 may be used as an evidence indicating how the assistant was assisting the patient. However, to protect privacy of the patient, the patient biological information of the patient stored in the storage apparatus 15 may be discarded with prescribed timing. In that situation, the timing with which the patient biological information of the patient is discarded may be the same as or may be different from the timing with which at least one of the image and the audio of the patient is discarded.

In the above description, the example was explained in which, when the assistant assists any of the patients doing the exercise for rehabilitation, the patient FB information is presented to the patient and/or the assistant FB information is presented to the assistant. However, another arrangement is also acceptable in which, when the assistant assists a subject performing an arbitrary action, FB information for assisting the arbitrary action is presented to at least one of the subject and the assistant. In other words, the rehabilitation assistance system SYS may be modified as appropriate so as to be applied to a situation where the assistant assists the subject performing the arbitrary action that is different from the rehabilitation. For example, when a teacher (the assistant) who is providing an education service assists a student (the subject) who is getting education, FB information for assisting the education of the student may be presented to at least one of the student and the teacher. In other words, the rehabilitation assistance system SYS may be modified into an education assistance system capable of presenting the FB information for assisting the education of the student to at least one of the student and the teacher. As another example, when a skilled worker (the assistant) who is highly skilled assists work of a non-skilled worker (the subject) who is not so highly skilled, FB information for assisting the work of the non-skilled worker may be presented to at least one of the non-skilled worker and the skilled worker. In other words, the rehabilitation assistance system SYS may be modified into an education assistance system capable of presenting the FB information for assisting the work of the non-skilled worker to at least one of the non-skilled worker and the skilled worker.

### (5) SUPPLEMENTARY NOTE

With respect to the example embodiments described above, the following Supplementary Notes will be further disclosed.

### Supplementary Note 1

A rehabilitation assistance apparatus including:
an acquisition unit configured to acquire at least one of an image of a subject who is doing exercise for rehabilitation, audio of the subject, and biological information of the subject;
a calculation unit configured to calculate a fatigue level of the subject based on at least one of the image, the audio, and the biological information;
a generation unit configured to generate feedback information related to the rehabilitation, based on the fatigue level; and
an output unit configured to output the feedback information.

### Supplementary Note 2

The rehabilitation assistance apparatus according to Supplementary Note 1, wherein the feedback information includes first feedback information for the subject.

### Supplementary Note 3

The rehabilitation assistance apparatus according to Supplementary Note 2, wherein
the first feedback information includes information for assisting the subject so that the subject does the exercise having an appropriate load.

### Supplementary Note 4

The rehabilitation assistance apparatus according to Supplementary Note 2 or 3, wherein
the first feedback information includes at least one of the following: exercise menu information related to a menu of the exercise presented to the subject; rest information related to proposing a rest to the subject; motivation increase information for increasing motivation of the subject doing the exercise; and advice information related to advice for the subject.

### Supplementary Note 5

The rehabilitation assistance apparatus according to any one of Supplementary Notes 1 to 4, wherein
when the fatigue level exceeds a first threshold value, the generation unit is configured to generate, as the feedback information, exercise menu information indicating a menu of an exercise having a load smaller than a load of the exercise currently presented to the subject.

### Supplementary Note 6

The rehabilitation assistance apparatus according to Supplementary Note 5, wherein
the first threshold value is set based on the menu of the exercise presented to the subject and the fatigue level.

### Supplementary Note 7

The rehabilitation assistance apparatus according to any one of Supplementary Notes 1 to 6, wherein
when the fatigue level is lower than a second threshold value, the generation unit is configured to generate, as the feedback information, exercise menu information indicating a menu of an exercise having a load larger than a load of the exercise currently presented to the subject.

### Supplementary Note 8

The rehabilitation assistance apparatus according to Supplementary Note 7, wherein the second threshold value is set based on the menu of the exercise presented to the subject and the fatigue level.

### Supplementary Note 9

The rehabilitation assistance apparatus according to any one of Supplementary Notes 4 to 8, wherein
when the fatigue level exceeds a third threshold value, the generation unit is configured to generate the rest information proposing a rest to the subject.

### Supplementary Note 10

The rehabilitation assistance apparatus according to Supplementary Note 9, wherein
the third threshold value is set based on the menu of the exercise presented to the subject and the fatigue level.

### Supplementary Note 11

The rehabilitation assistance apparatus according to any one of Supplementary Notes 1 to 10, wherein
the feedback information includes second feedback information for an assistant who assists the rehabilitation.

### Supplementary Note 12

The rehabilitation assistance apparatus according to Supplementary Note 11, wherein
the second feedback information includes information for assisting the assistant so that the assistant is able to cause the subject to do the exercise having an appropriate load.

### Supplementary Note 13

The rehabilitation assistance apparatus according to Supplementary Note 11 or 12, wherein
the second feedback information includes specific subject information indicating whether or not the fatigue level of the subject satisfies a prescribed condition.

### Supplementary Note 14

The rehabilitation assistance apparatus according to Supplementary Note 13, wherein
the prescribed condition includes at least one of a condition where the fatigue level exceeds a fourth threshold value and a condition where the fatigue level is lower than a fifth threshold value.

### Supplementary Note 15

The rehabilitation assistance apparatus according to Supplementary Note 14, wherein
each of the fourth and the fifth threshold values is set based on the menu of the exercise presented to the subject and the fatigue level.

### Supplementary Note 16

The rehabilitation assistance apparatus according to any one of Supplementary Notes 11 to 15, wherein
the acquisition unit is configured to acquire a plurality of images including the image and respectively showing a plurality of subjects including the subject, and
based on the images, the generation unit is configured to generate image information of the plurality of subjects as the second feedback information.

### Supplementary Note 17

The rehabilitation assistance apparatus according to any one of Supplementary Notes 11 to 16, wherein
the acquisition unit is configured to acquire a plurality of images including the image and respectively showing a plurality of subjects including the subject, and
based on the images, the generation unit is configured to generate, as the second feedback information, image information of the subjects corresponding to whether or not the fatigue levels satisfy a prescribed condition.

### Supplementary Note 18

The rehabilitation assistance apparatus according to any one of Supplementary Notes 11 to 17, wherein
the generation unit is configured to generate, as the second feedback information, image information of one or more of the subjects whose fatigue levels satisfy a prescribed condition; and image information that is of one or more of the subjects whose fatigue levels do not satisfy the prescribed condition and that is outputted in an output mode different from an output mode used for the image information of the one or more subjects whose fatigue levels satisfy the prescribed condition.

### Supplementary Note 19

The rehabilitation assistance apparatus according to Supplementary Note 18, wherein the output mode includes sizes of one or more images represented by the image information.

### Supplementary Note 20

The rehabilitation assistance apparatus according to any one of Supplementary Notes 11 to 19, wherein
the generation unit is configured to generate, as the second feedback information, advice input screen information used by the assistant for inputting advice for the subject.

### Supplementary Note 21

The rehabilitation assistance apparatus according to Supplementary Note 20, wherein
the advice input screen information includes information related to a plurality of mutually-different advice candidates.

### Supplementary Note 22

The rehabilitation assistance apparatus according to Supplementary Note 20 or 21, wherein
the generation unit is configured to generate, as the feedback information, advice information for the subject in accordance with an input from the assistant.

### Supplementary Note 23

The rehabilitation assistance apparatus according to any one of Supplementary Notes 1 to 22, wherein
the generation unit is configured to generate information related to the fatigue level as the feedback information, and
the output unit is configured to output the information related to the fatigue level.

### Supplementary Note 24

The rehabilitation assistance apparatus according to any one of Supplementary Notes 1 to 23, wherein
the fatigue level is an index value indicating a degree of physical fatigue of the patient caused by the exercise.

### Supplementary Note 25

The rehabilitation assistance apparatus according to any one of Supplementary Notes 1 to 24, further including:
a notification unit configured, when the subject is determined to be in the abnormal state based on at least one of the image, the audio, and the biological information, to notify a prescribed notification destination that the subject is in the abnormal state.

### Supplementary Note 26

The rehabilitation assistance apparatus according to any one of Supplementary Notes 1 to 25, wherein
in addition to the feedback information, the output unit is configured to output image information in an output mode that protects privacy of the subject.

### Supplementary Note 27

A rehabilitation assistance system including:
a subject terminal usable by a subject who is doing exercise for rehabilitation;
an assistant terminal usable by an assistant who assists the rehabilitation; and
a rehabilitation assistance apparatus, wherein
the rehabilitation assistance apparatus includes:
   an acquisition unit configured to acquire at least one of an image of the subject, audio of the subject, and biological information of the subject;
   a calculation unit configured to calculate a fatigue level of the subject based on at least one of the image, the audio, and the biological information;
   a generation unit configured to generate feedback information related to the rehabilitation, based on the fatigue level; and
   an output unit configured to output the feedback information to at least one of the subject terminal and the assistant terminal.

### Supplementary Note 28

A rehabilitation assistance method including:
acquiring at least one of an image of a subject who is doing exercise for rehabilitation, audio of the subject, and biological information of the subject;
calculating a fatigue level of the subject based on at least one of the image, the audio, and the biological information;
generating feedback information related to the rehabilitation, based on the fatigue level; and
outputting the feedback information.

### Supplementary Note 29

A recording medium having recorded thereon a computer program configured to cause a computer to execute:
acquiring at least one of an image of a subject who is doing exercise for rehabilitation, audio of the subject, and biological information of the subject;
calculating a fatigue level of the subject based on at least one of the image, the audio, and the biological information;
generating feedback information related to the rehabilitation, based on the fatigue level; and
outputting the feedback information.

It is possible to change the present invention as appropriate, so long as no conflict occurs with the gist or concept of the invention as understood from the claims and the entire specification. A Rehabilitation assistance apparatus, a rehabilitation assistance system, a rehabilitation assistance method, and computer program derived from such changes are also included in the technical concept of the present invention.

### DESCRIPTION OF REFERENCE CODES

SYS Rehabilitation assistance system
1 Patient terminal
11 Camera
12 Microphone
13 Biological sensor
14 Computation apparatus
16 Communication apparatus
18 Output apparatus
2 Rehabilitation assistance server
21 Computation apparatus
211 Information acquisition unit
212 Fatigue level calculation unit
213 FB information generation unit
23 Communication apparatus
3 Assistant terminal
31 Computation apparatus
33 Communication apparatus
35 Output apparatus

What is claimed is:

1. A rehabilitation assistance apparatus comprising:
at least one memory configured to store instructions; and
at least one processor configured to execute the instructions to:
acquire at least one of an image of a subject who is doing exercise for rehabilitation, audio of the subject, and biological information of the subject;
calculate a fatigue level of the subject based on at least one of the image, the audio, and the biological information;
generate feedback information related to the rehabilitation, based on the fatigue level; and
output the feedback information.

2. The rehabilitation assistance apparatus according to claim 1, wherein
the feedback information includes first feedback information for the subject,
the first feedback information includes information for assisting the subject so that the subject does the exercise having an appropriate load.

3. (canceled)

4. The rehabilitation assistance apparatus according to claim 2, wherein
the first feedback information includes at least one of the following: exercise menu information related to a menu of the exercise presented to the subject; rest information related to proposing a rest to the subject; motivation increase information for increasing motivation of the subject doing the exercise; and advice information related to advice for the subject.

5. The rehabilitation assistance apparatus according to claim 1, wherein
when the fatigue level exceeds a first threshold value, at least one processor configured to execute the instructions to generate, as the feedback information, exercise menu information indicating a menu of an exercise having a load smaller than a load of the exercise currently presented to the subject,
the first threshold value is set based on the menu of the exercise presented to the subject and the fatigue level.

6. (canceled)

7. The rehabilitation assistance apparatus according to claim 1, wherein
when the fatigue level is lower than a second threshold value, at least one processor configured to execute the instructions to generate, as the feedback information, exercise menu information indicating a menu of an exercise having a load larger than a load of the exercise currently presented to the subject,
the second threshold value is set based on the menu of the exercise presented to the subject and the fatigue level.

8. (canceled)

9. The rehabilitation assistance apparatus according to claim 4, wherein
when the fatigue level exceeds a third threshold value, at least one processor configured to execute the instructions to generate the rest information proposing a rest to the subject,
the third threshold value is set based on the menu of the exercise presented to the subject and the fatigue level.

10. (canceled)

11. The rehabilitation assistance apparatus according to claim 1, wherein
the feedback information includes second feedback information for an assistant who assists the rehabilitation,
the second feedback information is information for assisting the assistant so that the assistant is able to cause the subject to do the exercise having an appropriate load.

12. (canceled)

13. The rehabilitation assistance apparatus according to claim 11, wherein
the second feedback information includes specific subject information indicating whether or not the fatigue level of the subject satisfies a prescribed condition,
the prescribed condition includes at least one of a condition where the fatigue level exceeds a fourth threshold value and a condition where the fatigue level is lower than a fifth threshold value.

14. (canceled)

15. The rehabilitation assistance apparatus according to claim 14, wherein
each of the fourth and the fifth threshold values is set based on the menu of the exercise presented to the subject and the fatigue level.

16. The rehabilitation assistance apparatus according to claim 11, wherein
at least one processor configured to execute the instructions to acquire a plurality of images including the image and respectively showing a plurality of subjects including the subject, and
based on the images, at least one processor configured to execute the instructions to generate image information of the plurality of subjects as the second feedback information.

17. The rehabilitation assistance apparatus according to claim 11, wherein
at least one processor configured to execute the instructions to acquire a plurality of images including the image and respectively showing a plurality of subjects including the subject, and
based on the images, at least one processor configured to execute the instructions to generate, as the second feedback information, image information of the subjects corresponding to whether or not the fatigue levels satisfy a prescribed condition.

18. The rehabilitation assistance apparatus according to claim 11, wherein
at least one processor configured to execute the instructions to generate, as the second feedback information, image information of one or more of the subjects whose fatigue levels satisfy a prescribed condition; and image information that is of one or more of the subjects whose fatigue levels do not satisfy the prescribed condition and that is outputted in an output mode different from an output mode used for the image information of the one or more subjects whose fatigue levels satisfy the prescribed condition.

19. (canceled)

20. The rehabilitation assistance apparatus according to claim 11, wherein
the generation unit is configured to generate, as the second feedback information, advice input screen information used by the assistant for inputting advice for the subject.

21. The rehabilitation assistance apparatus according to claim 20, wherein
the advice input screen information includes information related to a plurality of mutually-different advice candidates.

22. The rehabilitation assistance apparatus according to claim 20, wherein
the generation unit is configured to generate, as the feedback information, advice information for the subject in accordance with an input from the assistant.

23. (canceled)

24. (canceled)

25. The rehabilitation assistance apparatus according to claim 1, further comprising:
a notification unit configured, when the subject is determined to be in the abnormal state based on at least one of the image, the audio, and the biological information, to notify a prescribed notification destination that the subject is in the abnormal state.

26. The rehabilitation assistance apparatus according to claim 1, wherein
in addition to the feedback information, the output unit is configured to output image information in an output mode that protects privacy of the subject.

27. (canceled)

28. A rehabilitation assistance method comprising:
acquiring at least one of an image of a subject who is doing exercise for rehabilitation, audio of the subject, and biological information of the subject;
calculating a fatigue level of the subject based on at least one of the image, the audio, and the biological information;
generating feedback information related to the rehabilitation, based on the fatigue level; and
outputting the feedback information.

29. A non-transitory recording medium having recorded thereon a computer program configured to cause a computer to execute:
acquiring at least one of an image of a subject who is doing exercise for rehabilitation, audio of the subject, and biological information of the subject;
calculating a fatigue level of the subject based on at least one of the image, the audio, and the biological information;
generating feedback information related to the rehabilitation, based on the fatigue level; and
outputting the feedback information.
